(19) United States
(12) Patent Application Publication
(10) Pub. No.: US 2013/0101574 A1
(43) Pub. Date: Apr. 25, 2013

(54) METHODS OF USING THROMBIN PEPTIDE DERIVATIVES

(71) Applicant: Darrell H. Carney, Dickinson, TX (US)

(72) Inventor: Darrell H. Carney, Dickinson, TX (US)

(21) Appl. No.: 13/711,291

(22) Filed: Dec. 11, 2012

Related U.S. Application Data
(63) Continuation-in-part of application No. PCT/US2011/040006, filed on Jun. 10, 2011.
(60) Provisional application No. 61/354,067, filed on Jun. 11, 2010.
(60) Provisional application No. 61/570,633, filed on Dec. 14, 2011.
(60) Provisional application No. 61/570,622, filed on Dec. 14, 2011.
(60) Provisional application No. 61/605,031, filed on Feb. 29, 2012.

(51) Int. Cl. A61K 38/46

(52) U.S. Cl. CPC A61K 38/465
USPC 424/94.6

(57) ABSTRACT

The present invention provides methods of reducing mortality in a subject exposed to a lethal dose of radiation, methods of reducing the risk of developing bacterial, fungal or viral systemic infection in a subject who is exposed or not exposed to radiation, methods of prevention and treatment of oral complications, particularly oral complications as a result of treatment with chemotherapy or radiation therapy, methods of treating dermal ulcers, particularly diabetic dermal ulcers, pressure dermal ulcers, venous stasis ulcers, and arterial ulcers, methods of promoting bone growth, and methods of promoting cartilage growth and repair in a subject in need thereof, by administering a thrombin peptide derivative to a subject. The administration may be local or systemic.

### RELATED APPLICATIONS

This application is a continuation-in-part of International Application No. PCT/US2011/040006, filed Jun. 10, 2011, published in the English language on Dec. 15, 2011 as International Publication No. WO 2011/156729, which claims the benefit of U.S. Provisional Patent Application No. 61/354,067, filed Jun. 11, 2010, both of which are incorporated by reference herein in their entirety. This application also claims the benefit of U.S. Provisional Application No. 61/570,633, filed Dec. 14, 2011, U.S. Provisional Application No. 61/570,622, filed Dec. 14, 2011, and U.S. Provisional Application No. 61/605,031, filed Feb. 29, 2012, all of which are incorporated by reference herein in their entirety.

### BACKGROUND OF THE INVENTION

With increasing threat of a nuclear detonation, it is essential to develop new countermeasures that can be delivered post-exposure to protect civilians and immediate care providers. Further urgency is mandated by the realization that a combination of radiation with traumatic injury, dermal injury or burns can be up to ten times more lethal than radiation alone. A detonation will injure thousands of people, who, without an effective countermeasure for combined radiation injury, will likely die from what should have been a sub-lethal dose of radiation.

In addition, more than 50% of all cancer patients undergo some degree of radiation therapy. It is well known that radiation therapy affects adjacent normal tissue, often preventing closure of surgical wounds and leading to later breakdown of skin or formation of chronic ulcers that fail to heal.

Currently, there are no products that have been approved for mitigating effects of radiation on individuals after radiation exposure. A number of potential products that are being evaluated only target a particular aspect of radiation combined injury, and therefore, have limited efficacies. For radiotherapy related injuries, most treatments are largely based on good wound care with the use of standard antibiotics and wound dressings with surgical repair of larger ulcerated or non-healing areas. The only FDA approved radiotherapy protective agent, Amifostin, however, is required to be injected into adjacent tissues prior to fractionated radiotherapy. Therefore, a need exists for new methods for preventing and treating radiation induced injuries resulted from accidental radiation exposure or radiotherapy.

Mucositis is the painful inflammation and ulceration of the mucous membranes lining the digestive tract, usually as an adverse effect of chemotherapy and radiotherapy treatment for cancer. Oral and gastrointestinal mucositis can affect up to 100% of patients undergoing high-dose chemotherapy and hematopoietic stem cell transplantation, 80% of patients with malignancies of the head and neck receiving radiotherapy, and a wide range of patients receiving chemotherapy. Alimentary track mucositis increases mortality and morbidity and contributes to rising health care costs. Mucositis can occur anywhere along the gastrointestinal (GI) tract, but oral mucositis refers to the particular inflammation and ulceration that occurs in the mouth. As a result of cell death in reaction to chemotherapy or radiation therapy, the mucosal lining of the mouth becomes thin, may slough off and become red, inflamed, ulcerated, and painful. Peripheral erythema is usually present. The degree of pain is usually related to the extent of the tissue damage. Due to pain, the patient may experience trouble speaking, eating, or even opening the mouth. Further, ulcers can result in both oral and systemic infection, including blood infection. Gastrointestinal mucositis can result in digestive difficulties.

Salivary gland dysfunction, also known as xerostomia, is also a common side effect observed with chemotherapy and radiation therapy. Xerostomia is characterized by dryness of the mouth due to thickened, reduced, or absent salivary flow; increased risk of infection and compromised speaking, chewing, and swallowing. Medications other than chemotherapy can also cause salivary gland dysfunction. Persistent dry mouth increases the risk for dental caries. Xerostomia can resolve upon completion of the medical intervention that caused the xerostomia, e.g., chemotherapy or radiation therapy, however, in some individuals, it becomes a chronic disorder.

Oral complications such as mucositis and xerostomia can become dose limiting toxicities in the treatment of cancer. Presently available treatments are largely palliative, e.g., analgesia, or to treat or prevent infections that can be associated with the conditions.

Dermal ulcers are associated with a number of conditions, including both type 1 and type 2 diabetes, and can result in significant treatment costs, as well as increased mortality and morbidity. Diabetes is associated with both poor circulation and peripheral neuropathies which permit the development of chronic diabetic ulcers which can result in infections including osteomyelitis (bone infections), which can result in the need for hospitalization and amputation.

Pressure ulcers are a highly prevalent condition that occur most often in individuals with limited mobility can be further exacerbated in the elderly and those with circulatory problems, chronically ill, or have decreased sensation or partial paralysis. Pressure ulcers arise from prolonged tissue ischemia caused by pressure that exceeds the tissue capillary pressure. Friction caused when moving patients to prevent the formation of pressure ulcers can further weaken the skin, potentially promoting or exacerbating the formation of ulcers. Further, the periods of immobility do not need to be very long to lead to the formation of pressure ulcers. For example, making fewer than 20 movements per night increases the risk of developing pressure ulcers, and the chance of developing pressure ulcers doubles in surgeries of greater than 4 hours in duration.

Ulcers can also occur as a result of poor circulation including venous stasis ulcers and arterial ulcers. Venous stasis ulcers are thought to arise when venous valves that exist to prevent backflow of blood do not function properly, causing the pressure in veins, particularly in the lower legs, to increase and blood to pool. Venous stasis ulcers typically occur in the legs, particularly the lower legs. Arterial ulcers, also known as ischemic ulcers, are mostly located on the lateral surface of the ankle or the distal digits. The ulcers are caused by lack of blood flow to the capillary beds of the lower extremities. When pressure is placed on the skin, the skin is damaged and is unable to be repaired due to the lack of blood perfusing the tissue. The wound has a characteristic deep, punched out look, often extending down to the tendons. Both types of ulcers can be quite painful.

In 1991, a market study was performed to estimate the treatment costs and the costs of hospital stays for patients who developed pressure ulcers during hospitalization. The cost were estimated to be as much as $6 billion a year. In elderly populations and in those who are institutionalized, pressure ulcers can be one of the most costly diseases to treat (see., e.g. emedicine.medscape.com/article/319284-overview). The cost of treatment was estimated to be about $2000-$40,000 per ulcer, depending on the stage of development, in addition to the cost in human suffering.

Similarly, poor circulation at the surgical site can prevent the proper healing of surgical wounds. Poor circulation can be due to a preexisting condition in the subject (e.g., diabetes, vascular complications, cancer, corticosteroid drugs), or the location or type of surgery (e.g., colon repair, joint replacement, tendon surgery, hysterectomy).

Mammalian bone tissue has a remarkable ability to regenerate and thereby repair injuries and other defects. For example, bone growth is generally sufficient to bring about full recovery from most simple and hairline fractures. However, this is not the case in all populations. Moreover, the rate of healing and regeneration may be slower than desired in some subjects and situations, even in normal individuals.

Further, there are many injuries, defects or conditions where bone growth is inadequate to achieve an acceptable outcome. For example, bone regeneration generally does not occur throughout large voids or spaces. Therefore, fractures cannot heal unless the pieces are in close proximity. If a significant amount of bone tissue is lost as a result of the injury, the healing process may be incomplete, resulting in undesirable cosmetic and/or mechanical outcomes. This is often the case with non-union fractures or with bone injuries resulting from massive trauma. Tissue growth is also generally inadequate in voids and segmental gaps in bone caused, for example, by surgical removal of tumors or cysts. In other instances, it may be desirable to stimulate bone growth where bone is not normally found, e.g., ectopically. Spine fusion to relieve lower back pain where two or more vertebrae are induced to fuse is one example of desirable ectopic bone formation. Currently, such gaps or segmental defects require bone grafts for successful repair or gap filling. The development of effective bone graft substitutes would eliminate the need to harvest bone from a second surgical site for a graft procedure, thereby significantly reducing the discomfort experienced by the patient and risk of donor site healing complications.

Distraction osteogenesis is a surgical process used to reconstruct skeletal deformities and lengthen the long bones of the body. A corticotomy is used to fracture the bone into two segments, and the two bone ends of the bone are gradually moved apart with an external fixatore during the distraction phase, allowing new bone to form in the gap. When the desired or possible length is reached, a consolidation phase follows in which the bone is allowed to keep healing. The process mimics a continuous fracture and healing situation. Distraction osteogenesis has the benefit of simultaneously increasing bone length and the volume of surrounding soft tissues. However, the procedure is lengthy and, as a result, prone to significant complications.

Unlike bone and most other tissues, cartilage does not self-repair following injury. Cartilage is an avascular tissue made up largely of cartilage specific cells, the chondrocytes, special types of collagen, and proteoglycans. The inability of cartilage to self-repair after injury, disease, or surgery is a major limiting factor in rehabilitation of degrading joint surfaces and injury to meniscal cartilage. Osteoarthritis, the major degenerative disease of weight bearing joint surfaces, is caused by eroding or damaged cartilage surfaces and is present in approximately 25% of the over 50-year-old population. In the US more than 20 million people suffer from osteoarthritis, with annual healthcare costs of more than $8.6 billion. In addition, the cost for cartilage repair from acute joint injury (meniscal lesions, patellar surface damage and chondromalacia) exceeds $1 billion annually. Therefore, new therapeutic approaches are needed to heal lesions of cartilage caused by degeneration or acute trauma.

### SUMMARY OF THE INVENTION

Applicants have discovered that post-exposure injection of the thrombin peptide derivative TP508 can increase survival time and delay onset of septic bacterial growth in mice that were exposed to a lethal dose of gamma irradiation (Examples 3 and 4). In addition, either topical treatment or systemic injection of the thrombin peptide derivative TP508 can promote healing of an open dermal wound in mice that were exposed to radiation (Example 5).

The present invention is directed to a method of reducing the risk of mortality or extending the life expectancy (by, e.g., at least 5%, 10%, 20%, 25%, 50%, 75% or 100%) in a subject exposed to a lethal dose of radiation, or to a dose of radiation that when combined with injury would be lethal, comprising administering to the subject an effective amount of a thrombin peptide derivative comprising Asp-Ala-R, wherein R is a serine esterase conserved sequence.

In another embodiment, the present invention is directed to a method of reducing the risk of developing systemic bacterial, fungal or viral infection in a subject exposed to radiation. The method comprises administering to the subject an effective amount of a thrombin peptide derivative comprising Asp-Ala-R, wherein R is a serine esterase conserved sequence.

In another embodiment, the present invention is directed to a method of treating a subject with traumatic injury, dermal injury and/or burn injury who is also exposed to radiation, comprising administering to the subject an effective amount of a thrombin peptide derivative, wherein the thrombin peptide derivative comprises Asp-Ala-R, wherein R is a serine esterase conserved sequence. The radiation exposure may be lethal, or may be sub-lethal.

The present invention is also directed to a method of reducing radiation related injury in a subject undergoing radiation therapy, comprising administering to the subject an effective amount of a thrombin peptide derivative, wherein the thrombin peptide derivative comprises Asp-Ala-R, wherein R is a serine esterase conserved sequence.

In another embodiment, the present invention is directed to a method of reducing the risk of developing a radiation induced illness in a subject undergoing radiation therapy, comprising administering to the subject an effective amount of a thrombin peptide derivative, wherein the thrombin peptide derivative comprises Asp-Ala-R, wherein R is a serine esterase conserved sequence.

In another embodiment, the present invention is directed to a method of promoting healing of a wound on a subject that was caused by radiation exposure and/or has been exposed to radiation, comprising administering to the wound an effective amount of a thrombin peptide derivative, or comprising administering an effective amount of a thrombin peptide derivative systemically post radiation exposure, wherein the thrombin peptide derivative comprises Asp-Ala-R, wherein R is a serine esterase conserved sequence.

In yet another embodiment, the present invention is directed to a method of reducing the risk of developing bacterial, fungal or viral infection in the blood of a subject that has not been exposed to radiation and that may be at risk of developing bacterial, fungal or viral infection in the blood. The method comprises administering to the subject an effective amount of a thrombin peptide derivative comprising Asp-Ala-R, wherein R is a serine esterase conserved sequence.

The present invention is also directed to the use of a thrombin peptide derivative for reducing the risk of mortality in a subject exposed to a lethal dose of radiation, reducing the risk of developing bacterial, fungal or viral infection in a subject exposed to radiation, treating a subject with traumatic injury, dermal injury and/or burn injury who is also exposed to radiation, reducing radiation related injury in a subject undergoing radiation therapy, reducing the risk of developing a radiation induced illness in a subject undergoing radiation therapy; promoting healing of a wound on a subject that was caused by radiation exposure and/or has been exposed to radiation, wherein the thrombin peptide derivative comprises Asp-Ala-R, wherein R is a serine esterase conserved sequence. For the use for promoting healing of a wound on a subject that was caused by radiation exposure and/or has been exposed to radiation.

In one embodiment, the invention provides compositions and methods for the prevention and treatment of mucositis and xerostomia caused by therapeutic interventions including, but not limited to, chemotherapy and radiation therapy for the treatment of cancer.

In one embodiment, the invention provides methods of preventing or treating oral complications in a subject associated with treatment with a chemotherapeutic agent or radiation in a subject undergoing treatment, comprising administering a thrombin peptide derivative to the subject, wherein the thrombin peptide derivative comprises Asp-Ala-R, wherein R is a serine esterase conserved sequence.

In certain embodiments, the oral complication is mucositis or xerostomia. In certain embodiments, the oral complication is characterized by at least one symptom selected from the group consisting of oral infection, bleeding, pain, taste alteration, nutritional compromise, thermal sensitivity, and abnormal dental development. In certain embodiments, the oral complication is an infection such as a bacterial infection, fungal infection, and viral infection. In certain embodiments, the subject is a pediatric subject and the symptom is abnormal dental development.

The invention provides methods of prevention or treatment of mucositis in a subject treated with a chemotherapeutic agent or radiation comprising administering a thrombin peptide derivative to the subject, wherein the thrombin peptide derivative comprises Asp-Ala-R, wherein R is a serine esterase conserved sequence. In certain embodiments, the thrombin peptide derivative is administered to the subject before, during, and/or after treatment with a chemotherapeutic agent. In certain embodiments, the thrombin peptide derivative is administered to the subject before, during, and/or after treatment with radiation. In certain embodiments, the radiation is administered to the head, neck, and/or gastrointestinal (GI) tract of the subject. In certain embodiments, the radiation is whole body radiation. In certain embodiments, the mucositis is GI mucositis. In other embodiments, the mucositis is oral mucositis. In certain embodiments, the chemotherapy is high dose chemotherapy.

The invention provides methods of prevention or treatment of xerostomia in a subject comprising administering a thrombin peptide derivative to the subject, wherein the thrombin peptide derivative comprises Asp-Ala-R, wherein R is a serine esterase conserved sequence. In certain embodiments, the subject is treated with a chemotherapeutic agent and/or radiation. In certain embodiments, the thrombin peptide derivative is administered to the subject before, during, and/or after treatment with a chemotherapeutic agent. In certain embodiments, the thrombin peptide derivative is administered to the subject before, during, and/or after treatment with radiation. In certain embodiments, radiation comprises radiation of at least a part of one or more body parts selected from the group consisting of head and neck. In certain embodiments, xerostomia is chronic xerostomia.

The thrombin peptide derivative can be administered locally or systemically. In certain embodiments, the thrombin peptide derivative is administered topically, sublingually, or bucally. For example, in certain embodiments, the thrombin peptide derivative is administered in a formulation such as a mouthwash, a gargle, a lozenge, a gum, a dissolvable film, a dissolvable tablet, and an oral coating formulation. In certain embodiment, an analgesic agent or an anti-inflammatory agent is also administered to the subject.

The invention provides pharmaceutical compositions comprising a thrombin peptide derivative wherein the thrombin peptide derivative comprises Asp-Ala-R, wherein R is a serine esterase conserved sequence, wherein the pharmaceutical composition is a composition selected from the group consisting of a mouthwash, a gargle, a lozenge, a gum, a dissolvable film, a dissolvable tablet, and an oral coating formulation. In certain embodiments, the pharmaceutical composition further includes an analgesic agent or an anti-inflammatory agent.

In one embodiment, the invention provides methods for the treatment of dermal ulcers, particularly chronic dermal ulcers associated with either type 1 or type 2 diabetes, pressure ulcers, venous stasis ulcers, and arterial ulcers. The invention also provides methods to treat surgical wounds, particularly poorly vascularized surgical wounds.

The invention provides method of treating a dermal ulcer in a subject comprising administering a thrombin peptide derivative to the subject, wherein the thrombin peptide derivative comprises Asp-Ala-R, wherein R is a serine esterase conserved sequence, or any other thrombin derived peptide provided herein, wherein the thrombin derived peptide is delivered systemically.

In certain embodiments, the dermal ulcer is a diabetic ulcer. In certain embodiments, the subject has type 1 diabetes or type 2 diabetes. In certain embodiments, the diabetic ulcer is on the subject on a portion of leg below the knee.

In certain embodiments, the dermal ulcer is a pressure ulcer. In certain embodiments, the pressure ulcer is on the ischium, sacrum, trochanter, or heel.

In certain embodiments, the dermal ulcer is a venous stasis ulcer, which may be present on the subject on a portion of leg below the knee.

In certain embodiments, the dermal ulcer is an arterial ulcer. In certain embodiments, the arterial ulcer is on the subject on a lateral surface of the ankle or the distal digits.

The invention provides for treating a dermal ulcer that is a chronic dermal ulcer.

The invention further provides methods of treating a surgical wound in a subject comprising administering a thrombin peptide derivative to the subject, wherein the thrombin peptide derivative comprises Asp-Ala-R, wherein R is a serine esterase conserved sequence, or any other thrombin peptide derivative provided herein, wherein the thrombin derived peptide is delivered systemically. In one embodiment, the surgical wound is from hysterectomy, vaginal surgery, lumpectomy, mastectomy, joint replacement, meniscal surgery, organ transplant, colonectomy, tendon repair, or ACL replacement.

In certain embodiment, the surgical wound is a slow healing wound. In certain embodiments, the subject with the surgical wound suffers from one or more conditions selected from the group consisting of diabetes, rheumatoid arthritis, vascular insufficiency, vascular disease, cancer, leukemia, and inflammatory disease. In certain embodiments, the subject with the surgical wound has been or is being treated with one or more therapeutic agents selected from the group consisting of corticosteroids, anti-asthmatic agents, COPD treatment agents, chemotherapeutic agents, and anti-inflammatory agents. In preferred embodiments, the surgical wound is poorly vascularized, either due to the nature of the tissue in the body, or due to a particular disease or condition in the subject, or the treatment of the subject with various therapeutic agents.

In a preferred embodiment of the invention, the thrombin peptide derivative is delivered systemically by injection. In certain embodiments the thrombin peptide derivative is delivered systemically by a non-topical parenteral route such as subcutaneous, intramuscular, intravenous, intradermal, intranasal, transcutaneous, infusion, and mucosal.

In certain embodiments, the thrombin peptide derivative is administered alone, for example in the treatment of a dermal wound or a surgical wound. In a preferred embodiment, the thrombin peptide derivative is not administered with a protease inhibitor to inhibit a protease at the ulcer site.

In certain embodiments, the thrombin peptide derivative is administered with an agent selected from the group consisting of analgesic agent, antimicrobial agent, disinfectant, and anti-inflammatory agents. In certain embodiments, the thrombin peptide derivative is administered with an angiogenic growth factor.

The invention provides methods of promoting bone growth and repair in a subject at a site in need thereof comprising systemically administering a thrombin peptide derivative to a subject. The invention provides methods of promoting cartilage growth and repair in a subject at a site in need thereof comprising systemically administering a thrombin peptide derivative to a subject.

The invention provides methods of stimulating bone growth at a site in a subject in need of bone growth, the methods comprising the step of systemically administering an effective amount of a thrombin peptide derivative to the subject, wherein the thrombin peptide derivative comprises Asp-Ala-R, wherein R is a serine esterase conserved sequence. In certain embodiments, the site in need of bone growth is in need of osteoinduction. In certain embodiments, the site is in need of osteoinduction is a bone graft, a segmental gap in a bone, a bone void, or at a non-union fracture, a spinal fusion site, a simple fracture, a site of bone surgery, a site of traumatic bone injury, or a site of distraction osteogenesis.

In certain embodiments, the subject is a normal subject. In certain embodiments, the subject is osteopenic. In certain embodiments, the subject has normal bone density. In certain embodiments, the subject has osteoporosis. In certain embodiments, the subject is suffering from osteogenesis imperfecta, is a post-menopausal woman, or is of advanced age.

The invention provides methods of stimulating cartilage growth or repair at a site in a subject in need of cartilage growth or repair, the method comprising the step of systemically administering an effective amount of a thrombin peptide derivative to the subject, wherein the thrombin peptide derivative comprises Asp-Ala-R, wherein R is a serine esterase conserved sequence. In certain embodiments, the site in need of cartilage growth or repair is an arthritic joint in a subject suffering from arthritis, wherein the arthritis is rheumatoid arthritis or osteoarthritis. In certain embodiments, the subject is being treated for cartilage damage or loss, for example due to traumatic or chronic injury. In certain embodiments, the subject is a normal subject. In certain embodiments, the subject suffers from one or more conditions selected from diabetes, advanced age, hypercholesterolemia, vascular disease, osteopenia, osteoporosis, and osteogenesis imperfecta.

The methods provided herein for growth or repair of bone of cartilage are preferably used on a subject that has not been exposed to excess radiation. In a preferred embodiment, the subject is human.

The methods of the present invention are directed at stimulating bone growth in a subject and can be used at sites of simple fractures or sites where bone growth would not occur, absent treatment with autologous bone grafts or administration of bone growth factors. The methods of the invention are also directed at stimulating cartilage growth or repair. The methods involve systemic administration of small peptides having homology to the segment between amino acid 508 and 530 of human prothrombin that are further discussed infra. These small peptides are inexpensive to prepare in bulk quantities and are osteoinductive at a low dose.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

### BRIEF DESCRIPTION OF THE DRAWINGS

FIG. 1 is a graph that shows caspase activity measured in human microvascular endothelial cells (HMVEC) exposed to 0, 5, 10 or 20 Gy of radiation and treated with saline or 30 ug/ml TP508.

FIG. 2 is a graph that shows percent of mice surviving on different days after sustaining 8 Gy radiation exposure and a dermal excision, who have also received treatment with saline placebo or TP508 delivered topically or intravenously.

FIG. 3 is a graph that shows percent of mice surviving at different days after receiving 12 Gy radiation exposure and a single post-exposure bolus dose of either saline or TP508.

FIG. 4 is a bar graph that shows the number of live bacteria (CFU) in the blood of mice 6 and 7 days after exposure to 0 or 12 Gy radiation and either a saline placebo or TP508 injection.

FIG. 5 is a bar graph that shows the rate of linear wound healing (measured as mm/day) in mice at days 0-5 and 5-16 post-exposure to 0 or 8 Gy irradiation and either saline placebo or TP508 applied topically or by intravenous injection.

FIG. 6 is a panel with two bar graphs. The bar graph in panel A shows serum IL-6 levels (ng/ml) measured 11 days post-irradiation in mice irradiated at 0 and 8 Gy and treated with either saline placebo (P) or TP508 administered either topically (TPt) or intravenously (TPiv). The bar graph in panel B shows serum IL-6 levels (ng/ml) measured 7 days post-irradiation in mice irradiated at 0 and 12 Gy and treated with either saline placebo (P) or TP508 administered either topically (TPt) or intravenously (TPiv).

FIG. 7 is a bar graph showing fold increase in the sprouting area after 5 days of aortic explant culture. The aortas were isolated from mice 24 hours after exposure to 0, 3, 8 or 10 Gy radiation and treatment with either saline placebo or TP508 administered intravenously.

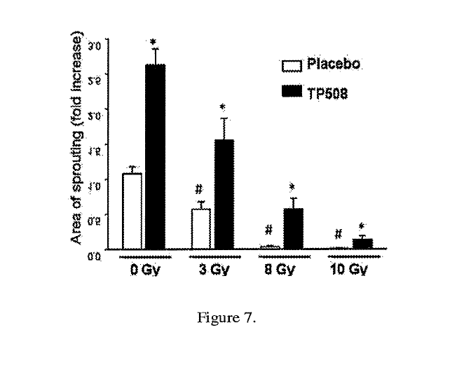

FIG. 8 is a bar graph showing the reduction in the number of live bacteria in normal and diabetic wounds. Chrysalin™ refers to TP508.

FIG. 9 is a graph that shows the effect of TP508 on promoting wound healing.

FIG. 10. “A” shows effects of systemic TP508 on angiogenesis under normoxic conditions. “B” shows effects of systemic TP508 on angiogenesis under hypoxic conditions. “C” shows the comparison of systemic TP508 on normal and VEGF-stimulated angiogenesis under normoxic and hypoxic conditions.

FIG. 11 shows the number of cells as a function of the level of CD45 expression.

FIG. 12 is a graph showing the percentage of cells expressing reduced levels of CD45 following TP508 injection.

### DETAILED DESCRIPTION OF ILLUSTRATIVE EMBODIMENTS

### Methods of Mitigating Effects of Radiation and Reducing the Risk of Systemic Infection

In one embodiment, the present invention is directed to methods of reducing the adverse effects of radiation exposure in a subject comprising administering to the subject an effective amount of a thrombin peptide derivative described therein. Radiation exposure can, for example, result from nuclear detonation, nuclear weaponry, accidental radiation exposure (such as due to accidents at nuclear reactors or inadequate protection from radiation source) or radiation therapy. Low-dose radiation exposure can be an occupational hazard affecting airline workers and astronauts; workers at nuclear power and nuclear fuel processing plants; research laboratory workers; and uranium miners. In addition, medical diagnostic tests, such as X-rays, can be a source of low-level radiation exposure for the general public.

The present invention is also directed to methods of reducing the risk of developing bacterial, fungal or viral infection in the blood in the subjects who have not been exposed to radiation, comprising administering to the subject an effective amount of a thrombin peptide derivative described herein. The infection can enter the bloodstream as a complication of diseases, such as pneumonia or meningitis, during surgery (especially when it involves mucous membranes such as the gastrointestinal tract), or due to catheters and other foreign bodies entering the arteries or veins (including intravenous drug abuse). In addition, individuals suffering from pulmonary conditions, inflammatory bowel disease and systemic inflammatory response syndrome (SIRS), or individuals who are immunocompromised are also at the risk of blood infection. In the hospital, indwelling catheters are a frequent cause of blood infections because they provide a means by which bacteria normally found on the skin can enter the bloodstream. Other causes of blood infections include dental procedures (occasionally including simple tooth brushing), herpes (including herpetic whitlow), urinary tract infections, peritonitis, Clostridium difficile colitis, intravenous drug use, and colorectal cancer. Blood infections may also be a consequence of oropharyngeal, gastrointestinal or genitourinary surgery or exploration. An immune response to blood infection can lead to sepsis and septic shock, which have a relatively high mortality rate. In the methods described herein, the peptide of the present invention can be administered in combination with an antibiotic.

In one aspect, the present invention is directed to a method of reducing the risk of mortality in a subject exposed to a lethal dose of radiation, comprising administering to the subject an effective amount of a thrombin peptide derivative described herein.

A lethal dose of radiation is a dose that would cause death in half of the tested subjects in 10 days, i.e. LD₅₀ in 10 days. The lethal dose depends on the identity of the subject. For example, a lethal dose for a human is about 3.5 Gy or greater and a lethal dose for a mouse is about 12 Gy or greater.

The subject who is exposed to a lethal dose of radiation may also have additionally sustained traumatic injury, dermal injury and/or burn injury.

A “burn injury” is a type of skin injury caused by heat, electricity, chemicals, light, radiation, friction or heat. Burn injury caused by radiation exposure, such as in the event of nuclear detonation, includes, for example, thermal burns from infrared heat radiation, beta burns from shallow ionizing beta radiation, and gamma burns from highly penetrating gamma radiation. The burn injury can be first-degree, second-degree or third-degree burn. Various percentage of total body surface area (TBSA) may be affected by the burn injury. For example, less than 1%, greater than 1%, greater than 5%, greater than 10%, greater than 15%, greater than 20%, greater than 30%, greater than 40%, greater than 50%, or 1-10%, 10-20%, 20-30%, 30-40%, 40-50% or 50-70% of TBSA was affected by the burn injury.

A “traumatic injury” is a physical injury produced by force or shock, for instance, a sudden injury caused by an extrinsic agent or force, e.g., vehicular accident, falling injury, blunt trauma, gunshot injury. A traumatic injury is often associated with secondary complications, such as shock, respiratory failure and death. For example, a traumatic injury can be caused by force of explosions or force of falling and flying objects. A traumatic injury also includes an injury to an internal organ resulting in hemorrhaging from the organ and/or at least partial loss of function. For example, an injury to an internal organ can be caused by penetration, such as from a bullet or flying projectile. A traumatic injury can also include injuries to musculoskeletal system, such as bones, muscles, cartilages, tendons, ligaments, joints and connective tissues. In one embodiment, a traumatic injury is a bone fracture.

Dermal injury is an injury to the dermis layer of the skin. Severe dermal injury is a dermal injury that results in a dermal wound that covers at least 100 mm² and/or is full-thickness wound (i.e., wound that penetrates through both the epidermis and dermis layer of the skin).

In one embodiment, the burn injury, traumatic injury or dermal injury, such as a severe dermal injury, sustained by the subject exposes the subject to systemic infection. “Systemic infection” is an infection that has entered the blood stream and may affect multiple organs and/or tissues or the body as a whole. In one embodiment, the present invention includes a method for treating a subject with a burn injury, traumatic injury or dermal injury, such as a severe dermal injury, who is exposed to radiation.

In another embodiment, the thrombin peptide derivatives described herein reduce leucocytopenia and/or neutropenia in the subject being treated by the methods of the present invention. Alternatively, the thrombin peptide derivatives described herein reduce the decrease in population of bone marrow progenitor cells from radiation damage in the subject being treated by the methods of the present invention.

In another aspect, the present invention is a method of reducing the risk of developing systemic bacterial, fungal or viral infection in a subject, comprising administering to the subject an effective amount of a thrombin peptide derivative described herein. In one embodiment, the subject has been exposed to a bacterial, fungal or viral infection. In one example, a subject who is at risk of developing systemic bacterial, viral or fungal infection has been exposed to radiation. For example, in the event of nuclear detonation, health workers, hospital patients, rescue workers, sanitarian workers and people who are in an environment or a place that is likely to have an outbreak of bacterial, fungal or viral infection, such as in a hospital. Alternatively, a subject who suffers from burn injury, traumatic injury or dermal injury resulting from radiation exposure, for example, from nuclear detonation, is more susceptible to bacterial, fungal or viral infection than a subject who does not suffer such injuries. In another alternative, a subject who has an unhealed wound prior to radiation exposure is more likely to develop bacterial, fungal or viral infection. In yet another alternative, a patient who is undergoing radiotherapy and has a pre-existing wound that is exposed to the radiation, for example from a surgery, would have higher risk for developing bacterial, fungal or viral infection than a patient who does not have a pre-existing wound. The thrombin peptide derivatives described herein are effective in reducing the risk of developing systemic bacterial, fungal or viral infection in the subjects described above upon radiation exposure.

In one embodiment, the method reduces the risk of developing bacterial, fungal or viral infection in the blood of the subject. High doses of radiation exposure can result in acute illness, such as breakdown of intestinal walls, which would render the subject more susceptible to septic infection. In one embodiment, the present invention is directed to a method of delaying the onset of septic systemic infection in a subject who is exposed to radiation comprising administering to the subject an effective amount of a thrombin peptide derivative described herein. The subject may be exposed to a lethal dose of radiation. Alternatively, the subject is exposed to a sub-lethal dose of radiation.

In another alternative, the subject is at risk of developing bacterial, fungal or viral infection and has not been exposed to radiation. These subjects include one or more of the following: a) a subject has sustained traumatic injury, severe dermal injury and/or burn; b) a subject that underwent an invasive medical or dental procedure; c) a subject that underwent insertion of an invasive medical device; d) a subject who has pneumonia or other pulmonary conditions that could lead to acute respiratory distress syndrome or systemic infections; e) a subject who is immunocompromised; f) a subject who is an infant or is older than 60 years old, or g) subject from one or more of the categories a-d, who is an infant or older than 60 years old. In one embodiment, the subject is exposed to the infection during or within 30 days after an invasive medical or dental procedure.

An invasive medical procedure that exposes a subject to infection is any procedure that involves either making a surgical cut in the skin or inserting an instrument, such as a needle or a tube, into the body of a subject. An invasive medical procedure increases a risk of introducing foreign organisms, such as bacteria or fungi, into the body of a subject, leading to an increased risk of bacterial, fungal or viral infection in the blood of the subject. An example of an invasive medical procedure is a surgery for any indication. Another example of an invasive medical procedure is a procedure wherein an invasive medical devise is introduced into a subject. Examples of invasive medical devises can include an intravenous or an arterial line, a breathing tube, a urinary catheter, a surgical drain, an artificial joint, or a feeding tube. Examples of feeding tubes can include G-tube/PEG tube, J-tube (jejunostomy tube) and NG-tube (nasogastric tube).

In another embodiment, the present invention is directed to a method of reducing the risk of developing a bacterial, fungal or viral infection in the blood of a subject who has pneumonia. Pneumonia is an inflammatory condition of the lung, especially of the alveoli. Infection is the most common cause of pneumonia. Infecting agents can be bacteria, viruses, fungi, or parasites. Chemical burns or physical injury to the lungs can also produce pneumonia. Bacteria are the most common cause of pneumonia, with Streptococcus pneumoniae the most commonly isolated bacteria in the cases of community-acquired pneumonia. Another important Gram-positive cause of pneumonia is Staphylococcus aureus, with Streptococcus agalactiae being an important cause of pneumonia in newborn infants. Gram-negative bacteria cause pneumonia less frequently than gram-positive bacteria. Some of the gram-negative bacteria that cause pneumonia include Haemophilus influenzae, Klebsiella pneumoniae, Escherichia coli, Pseudomonas aeruginosa and Moraxella catarrhalis. These bacteria often live in the stomach or intestines and may enter the lungs if vomit is inhaled. “Atypical” bacteria which cause pneumonia include Chlamydophila pneumoniae, Mycoplasma pneumoniae, and Legionella pneumophila.

In another embodiment, the present invention is directed to a method of reducing the risk of developing a bacterial, fungal or viral infection in the blood of a subject who is immunocompromised. An immunocompromised subject is a subject whose immune system is weakened or absent. Subjects who are immunocompromised are less capable of battling infections because of an immune response that is not properly functioning. Examples of immunocompromised subjects can include: a) subjects who have genetic defects that can affect functioning of their immune systems; b) subjects who have diseases such as AIDS or cancers, including leukemia, lymphoma or multiple myeloma; c) subjects who have chronic diseases, such as end-stage renal disease requiring dialysis, diabetes, or cirrhosis; d) subjects who receive treatments that can include steroids, chemotherapy, radiation, immunosuppressive post-transplant medications; and e) subjects who are pregnant.

In one embodiment, the bacterial, fungal or viral infection includes (in the presence or absence of radiation exposure), but is not limited to, infection of staphylococci (e.g., staphylococcus aureus), enterococci, streptococci (e.g., streptococcus pneumoniae), pseudomonas aeruginosa, burkholderia cenocepacia, mycobacterium avium, enterobacter, bacteroides fragilis, streptococcus pyogenes, enterococcus sp., haemophilus influenzae, legionella sp., chlamydia pneumoniae, escherichia coli, clostridium sp., staphylococcus sp., enterobacter sp., proteus sp., neiserria meningitidis, listeria monocytogenes, Candida sp. (e.g., Candida albicans), enterococcus sp., klebsiella, s. agalactiae, and aspergillus. The bacterial, fungal or viral infection also includes systemic bacterial, systemic fungal infections and systemic viral infections.

In the event of nuclear detonation, people often suffer open dermal wounds in addition to radiation exposure. Additionally, radiation exposure can often cause skin injuries, such as skin ulceration. In the case of radiation therapy, cancer patients often undergo radiation therapy following surgical removal of the tumor and consequently have surgical wounds that are exposed to radiation when undergoing radiation therapy. A subject may also have a pre-existing wound before radiation exposure, including nuclear detonation, accidental radiation exposure or radiation therapy. The thrombin peptide derivatives described herein can promote healing of these wounds described above.

In one embodiment, the radiation exposure is sub-lethal. For example, the radiation exposure is less than 3.5 Gy when the subject is a human.

In yet another aspect, the present invention is directed to a method of reducing radiation related injury in a subject who is undergoing a radiation therapy. The method comprises administering to the subject an effective amount of a thrombin peptide derivative described herein.

A “radiation related injury” is an injury due to radiation exposure resulting from nuclear detonation, nuclear weaponry, accidental radiation exposure or a radiation therapy. For example, when a subject is exposed to a high dose of radiation, such as in the event of nuclear detonation, the radiation exposure often causes acute illness in the subject, including hematopoietic syndrome as a result of effects of radiation on the bone marrow, spleen and lymph nodes, gastrointestinal syndrome (such as breakdown of the intestinal wall) due to the effects of radiation on the cells lining the digestive tract, and brain damage. Radiation exposure, such as radiation therapy, can also cause various skin injuries, such as intense reddening, blistering and ulceration of the skin at the irradiated site and late stage skin breakdown, and injury to hair follicles causing hair loss. Large dose of radiation exposure can cause permanent hair loss, damaged sebaceous and sweat glands, atrophy, fibrosis (e.g., subcutaneous fibrosis), decreased or increased skin pigmentation, and ulceration or necrosis of the exposed tissue. In one embodiment, the radiation related injury is skin ulceration or late stage breakdown. Radiation exposure, such as radiation therapy, can also damage musculoskeletal system, such as bones, muscles, cartilages, tendons, ligaments, joints and connective tissues. In one embodiment, the present invention is directed to a method of promoting healing of a bone in a subject who is exposed and/or has been exposed to radiation exposure, wherein the bone is a fractured bone or has been surgically treated, for example, to remove tumor in the bone.

In yet another aspect, the present invention is directed to a method of reducing the risk of developing a radiation induced illness in a subject undergoing radiation therapy, comprising administering to the subject an effective amount of a thrombin peptide derivative described herein.

A “radiation induced illness” refers to a disorder, disease or condition that resulted from cellular damages caused by radiation exposure. For example, exposure to radiation can result in various cellular damages, such as damages to hematopeotic cells, decreased availability, viability and function of progenitor cells, delayed angiogensis and revascularization, apoptosis of intestinal microvascular endothelial cells, epithelial cells, crypt cells, neuronal cells in the brain and other tissues, and myocardium. As such, radiation induced illnesses include diseases, disorders or conditions resulting from the above-described cellular damages caused by radiation exposure. Exemplary radiation induced illness include, but is not limited to, leucocytopenia, neutropenia, infections, systemic inflammatory response syndrome (SIRS), sepsis, multiple organ dysfunction syndrome (MODS), lung damage, lung/airway disease, brain microvascular damage, brain cerebrovascular damage, stroke, atherosclerosis, peripheral vascular damage, peripheral artery disease (PAD), diabetic neuropathy and angiopathy and cancer.

In yet another aspect, the present invention is directed to a method of promoting healing of a wound on a subject that was caused by radiation exposure and/or has been exposed to radiation. The method comprises administering to the wound (e.g., topically or systemically, such as by I.V.) an effective amount of a thrombin peptide derivative described herein.

A wound is a type of injury in which skin is torn, cut or punctured (an open wound). An open wound can include incisions or incised wounds (caused by a clean, sharp-edged object such as a knife, a razor or a glass splinter); lacerations (irregular tear-like wounds caused by blunt trauma); abrasions; puncture wounds; penetration wounds or gunshot wounds (caused by a bullet or similar projectile driving into or through the body).

As used herein, the thrombin derivative peptides, the modified thrombin peptide derivatives and the thrombin peptide derivative dimers described below can be collectively referred to as “thrombin peptide derivatives.” The thrombin derivative peptides, the modified thrombin peptide derivatives and each polypeptide in the thrombin peptide derivative have 19 to 23 amino acids (i.e., 19-23 amino acids in length).

### Prevention and Treatment of Oral Complications

In one embodiment, the invention provides compositions and methods for the prevention and treatment of oral complications associated with treatment of cancer, including chemotherapy and therapeutic radiation, by administration of a thrombin peptide derivative. The invention further provides compositions and methods for the prevention and treatment of mucositis and xerostomia by administration of a thrombin peptide derivative. The compositions and methods are particularly useful in the prevention and treatment of mucositis and xerostomia associated with the treatment of cancer using chemotherapy and/or radiation therapy. For example, the compositions can be used to prevent or treat one or more signs or symptoms of mucositis and xerostomia.

The treatment of cancer is frequently complicated by the limiting toxicities of the therapeutic interventions used, including chemotherapy and radiation therapy. For example, both treatment modalities can result in the development of oral complications such as mucositis which results in painful ulcerations throughout the gastrointestinal (GI) tract, particularly in the mouth, which can limit a patient's ability to eat and even drink in the most severe cases, resulting in nutritional deficiencies and wasting. Gastrointestinal mucositis can result in digestive difficulties such as nausea, vomiting, and diarrhea. Further, the persistent open sores provide an opportunity for infection as both chemotherapy and radiation therapy can result in immune suppression. Xerostomia, or salivary gland dysfunction, is another common oral complication of chemotherapy. Xerostomia can include dry mouth due to reduced or absent saliva production or thickened saliva which can result in eating difficulties and increased tooth decay. Head and neck radiation can cause permanent damage to the salivary glands resulting in chronic xerostomia that persists long after the termination of cancer treatment.

Treatment of cancer often involves multiple treatment modalities, e.g., chemotherapy, radiation, and surgery. As a result, the body is sequentially exposed to treatments that can result in undesirable side effects. Therefore, the compositions and methods provided herein can be used at any time during the treatment of a subject having cancer.

For example, certain treatment regimens including certain chemotherapeutic agents, high dose chemotherapy, and radiation for head and neck cancers (e.g., tongue cancer, throat cancer, brain cancer, thyroid cancer) result in a high incidence of mucositis or xerostomia, i.e., as high as 90%. In subjects undergoing such treatments who are at substantial risk of developing mucositis or xerostomia, the thrombin peptide derivative can be administered prophylactically and throughout the course of treatment. Alternatively, in certain therapeutic regimens for the treatment of cancer that do not include the use of high dose chemotherapy or radiation of the head and neck (e.g., low to moderate grade breast cancer or liver cancer), the incidence of mucositis and xerostomia is lower. In such treatment regimens, the subject can be observed for the development of one or more signs or symptoms of mucositis or xerostomia, and treated with the thrombin peptide derivative upon development of one or more signs or symptoms of the condition. In all cases, a thrombin peptide derivative can be administered as long as the subject is exposed to conditions that can cause mucositis or xerostomia, or as long as symptoms are present, particularly in the case of xerostomia which may become chronic after certain treatment regimens.

Therefore, the invention provides a method for identifying a subject susceptible to mucositis and treating to reduce the likelihood of the subject developing mucositis. Similarly, the invention provides method for identifying a subject susceptible to xerostomia and treating to reduce the likelihood of the subject developing xerostomia. The invention also provides methods for monitoring a subject for at least one sign or symptom of mucositis, and methods for monitoring a subject for at least one sign or symptom of xerostomia.

Mucositis and the presence of open sores increases the risk of infection, both locally and systemically. Therefore, in another aspect, the present invention provides methods for preventing or treating an infection in a subject who has undergone or is undergoing treatment for cancer who is suffering from mucositis, including administering to the subject a thrombin peptide derivative described herein. In one embodiment, the subject has been exposed to an infective agent such as a bacteria, fungus, or virus and is also suffering from chemotherapy associated mucositis. In another embodiment, the subject has been exposed to an infective agent such as a bacteria, fungus, or virus and is also suffering from radiation therapy associated mucositis. In certain embodiments, the infection is a local infection.

Infections can be caused by any infectious organism capable of infecting the subject treated with chemotherapy and/or radiation therapy, i.e. typically a human. In one embodiment, the bacterial, fungal or viral infection includes, but is not limited to, infection of staphylococci (e.g., staphylococcus aureus), enterococci, streptococci (e.g., streptococcus pneumoniae), pseudomonas aeruginosa, burkholderia cenocepacia, mycobacterium avium, enterobacter, bacteroides fragilis, streptococcus pyogenes, enterococcus sp., haemophilus influenzae, legionella sp., chlamydia pneumoniae, escherichia coli, clostridium sp., staphylococcus sp., enterobacter sp., proteus sp., neiserria meningitidis, listeria monocytogenes, Candida sp. (e.g., Candida albicans), enterococcus sp., klebsiella, s. agalactiae, aspergillus, Haemophilus influenzae, Klebsiella pneumoniae, Escherichia coli, Pseudomonas aeruginosa, Moraxella catarrhalis, Chlamydophila pneumoniae, Mycoplasma pneumoniae, and Legionella pneumophila.

As used herein, “oral complications associated with treatment of cancer” and the like, wherein treatment of cancer includes, for example, treatment with chemotherapy or radiation, or both. Oral complications associated with the treatment of cancer include, but are not limited to, mucositis and xerostomia and their associated symptoms including inflammation and ulceration of the mucous membranes lining the mouth, difficulties with or compromised drinking, eating, chewing, or swallowing, oral soft tissue pain (as distinguished from tooth pain), thermal sensitivity, dry mouth, thickening, reduction, or absence of saliva, increased dental cavities, infection due to the presence of ulcers and open sores, wasting, and nutritional deficiencies. Oral complications associated with the treatment of cancer can be associated with radiation, chemotherapy, or both. Oral complications associated with the treatment of cancer can be, but need not be, dose limiting toxicities. Oral complications associated with the treatment of cancer may resolve upon completion of the cancer treatment, or may persist, particularly with xerostomia, due to permanent damage of the salivary glands. Oral complications are characterized by the presence of at least one sign or symptom of oral infection including bacterial infection, fungal infection, and viral infection, bleeding, pain, taste alteration, nutritional compromise, or abnormal dental development.

As used herein, “mucositis” is understood as a condition characterized by the painful inflammation and ulceration of the mucous membranes lining the digestive tract, usually as an adverse effect of chemotherapy and radiotherapy treatment for cancer. Mucositis can be further characterized as oral or gastrointestinal mucositis, however, when not otherwise defined, mucositis should be understood to refer to either or both oral and gastrointestinal mucositis. Mucositis can occur anywhere along the gastrointestinal (GI) tract, but oral mucositis refers to the particular inflammation and ulceration that occurs in the mouth. Mucositis is characterized by thinning and sloughing off of the mucosal lining of the mouth. The mouth becomes red, inflamed, ulcerated, painful, excessively sensitive to heat or cold. Peripheral erythema is usually present. The degree of pain is usually related to the extent of the tissue damage. Due to pain, the patient may experience trouble speaking, eating, or even opening the mouth. Gastrointestinal mucositis can be accompanied by digestive dysfunction including nausea, vomiting, and diarrhea.

As used herein “xerostomia” is understood as salivary gland dysfunction that is not caused by a structural malformation of the salivary glands, but instead is caused by therapeutic interventions such as chemotherapy and radiation therapy, particularly of the head and neck, but may also occur to a lesser extent with other agents including some antidepressants. Xerostomia is characterized by dryness of the mouth due to thickened, reduced, or absent salivary flow; increased risk of infection and compromised speaking, chewing, and swallowing. Persistent dry mouth increases the risk for dental caries. Xerostomia can resolve upon completion of the medical intervention that caused the xerostomia, e.g., chemotherapy or radiation therapy, however, is some individuals, it becomes a chronic disorder.

As used herein, “high-dose chemotherapy” is understood as an intensive drug treatment to kill cancer cells, but that also destroys the bone marrow and can cause other severe side effects. High-dose chemotherapy is usually followed by bone marrow or stem cell transplantation to rebuild the bone marrow.

“Chemotherapeutic agents” as used herein is understood as any drug that kills a cancer cell and that can cause oral complications including mucositis and xerostomia. More specifically, chemotherapeutic agents include, but are not limited to classes of agents and agents such as fluorouracil, 5-FU, eloxatin, angiogenesis inhibitors such as erbitux and cetuximab; alkylating agents such as cisplatin and carboplatin, oxaliplatin, mechlorethamine, cyclophosphamide, chlorambucil, ifosfamide; anti-metabolites such as azathioprine or mercaptopurine; plant alkaloids such as vinca alkaloids (vincristine, vinblastine, vinorelbine, vindesine) and taxanes (paclitaxel); etoposide and teniposide; and topoisomerase inhibitors including topoisomerase I inhibitors including the camptothecins irinotecan and topotecan, and topoisomerase II inhibitors including amsacrine, etoposide, etoposide phosphate, and teniposide.

### Prevention and Treatment of Dermal Ulcers and Surgical Wounds

In one embodiment, the invention provides methods for the treatment of dermal ulcers by systemic administration of a thrombin peptide derivative. The methods are particularly useful in the treatment of dermal ulcers associated with type 1 or type 2 diabetes, pressure ulcers, venous stasis ulcers, and venous ulcers, particularly chronic dermal ulcers. For example, the compositions can be used to treat one or more signs or symptoms of dermal ulcers including chronic dermal ulcers. In a preferred embodiment, the compositions are administered by injection. The invention further provides methods of treating surgical wounds to promote healing, particularly surgical wounds at sites.

The treatment of diabetes is complicated by conditions caused by glucose imbalance, insulin deficiency, or insulin insensitivity. Poor circulation and peripheral neuropathies, both associated with diabetes, can result in the formation of chronic dermal ulcers, particularly in the feet and legs. Further, the persistent open sores provide an opportunity for infection, both local and systemic infection, particularly a bone infection. This results in a high cost of care and contributes to the mortality and morbidity of the disease.

Pressure ulcers are common in elderly and pediatric patients who spend significant amounts of time supine. Due to variations in methodologies and descriptions of the lesions, the incidence of pressure ulcers in hospitalized patients has been stated to be from 2.7% to 29% with a prevalence of 3.5% to 69%. There is a further increased risk for patients in critical care units and particular in the elderly admitted for non-elective orthopedic procedures, with an incidence of 66% (see., e.g. emedicine.medscape.com/article/319284-overview). Those with spinal cord injuries are also at significant risk for developing pressure ulcers. Common sites of occurrence include the ischium, sacrum, trochanter, and the heel.

Ulcers can also occur as a result of poor circulation including venous stasis ulcers and arterial ulcers. As the etiology of the diseases is not fully understood, interventions for the treatment of the conditions are somewhat limited. Both types of ulcers can be quite painful.

The invention provides methods for the treatment of a dermal ulcer in a subject susceptible to the development of a dermal ulcer. In certain embodiments, the method includes monitoring a subject for at least one sign or symptom of a chronic dermal ulcers. In certain embodiments, the ulcer is a diabetic ulcer. In certain embodiments, the ulcer is a chronic diabetic ulcer. In certain embodiments, the ulcer is a pressure ulcer. In certain embodiments, the ulcer is a venous stasis ulcer. In certain embodiments, the ulcer is an arterial ulcer.

In another aspect, the present invention is a method of treating an infection in a subject with a dermal ulcer, comprising administering to the subject a thrombin peptide derivative described herein. In one embodiment, the subject has been exposed to an infective agent such as a bacteria, fungus, or virus and is also has type 1 or type 2 diabetes. In one embodiment, the subject has been exposed to an infective agent such as a bacteria, fungus, or virus and also has a pressure ulcer. In one embodiment, the subject has been exposed to an infective agent such as a bacteria, fungus, or virus and is also a venous stasis ulcer. In one embodiment, the subject has been exposed to an infective agent such as a bacteria, fungus, or virus and has an arterial ulcer. In certain embodiments, the infection is a local infection. In certain embodiments, the local infection comprises an infection in a bone proximal to the ulcer.

Infections can be caused by any infectious organism capable of infecting a subject having, for instance, a dermal ulcer and/or a surgical wound, i.e. typically a human. In one embodiment, the bacterial, fungal or viral infection includes, but is not limited to, infection of staphylococci (e.g., staphylococcus aureus), enterococci, streptococci (e.g., streptococcus pneumoniae), pseudomonas aeruginosa, burkholderia cenocepacia, mycobacterium avium, enterobacter, bacteroides fragilis, streptococcus pyogenes, enterococcus sp., haemophilus influenzae, legionella sp., chlamydia pneumoniae, escherichia coli, clostridium sp., staphylococcus sp., enterobacter sp., proteus sp., neiserria meningitidis, listeria monocytogenes, Candida sp. (e.g., Candida albicans), enterococcus sp., klebsiella, s. agalactiae, aspergillus, Haemophilus influenzae, Klebsiella pneumoniae, Escherichia coli, Pseudomonas aeruginosa, Moraxella catarrhalis, Chlamydophila pneumoniae, Mycoplasma pneumoniae, and Legionella pneumophila.

“Ulcers” as used herein is understood as a break in skin or mucous membrane with loss of surface tissue, disintegration and necrosis of epithelial tissue, and often pus that are present at the site of tissue disruption. An ulcer is also a break in the skin or mucous membrane that does not respond to treatment using routine wound care.

As used herein, “dermal ulcers” include all ulcers present in the skin including diabetic ulcers, pressure ulcers, venous stasis ulcers, and arterial ulcers.

“Chronic” as in “chronic ulcer” is understood as being marked by a long duration or frequent recurrence. As used herein, a chronic ulcer is understood to be an ulcer that does not heal in an orderly set of stages and in a predictable amount of time the way most wounds do. Ulcers that do not heal within three months are often considered chronic. Chronic ulcers seem to be detained in one or more of the phases of wound healing. For example, chronic ulcers often remain in the inflammatory stage for too long. In acute wounds, there is a precise balance between production and degradation of molecules such as collagen; in chronic ulcers this balance is lost and degradation plays too large a role. Chronic ulcers may never heal or may take years to do so. These ulcers cause patients severe emotional and physical stress and create a significant financial burden on patients and the whole healthcare system.

“Diabetic ulcers”, also known as “neurogenic ulcers” or “neuropathic ulcers”, are understood as a break in skin or mucous membrane with loss of surface tissue, disintegration and necrosis of epithelial tissue, and often pus that are present on a person suffering from diabetes. Those suffering from diabetes are predisposed to peripheral neuropathy, which results in a decreased or total lack of sensation in the feet, and poor circulation. This results in a lack of awareness of injury combined with decreased healing. Therefore, a small cut or irritation can develop into an ulcer in the absence of proper awareness or care.

As used herein, “pressure ulcers” are understood as a break in skin or mucous membrane with loss of surface tissue, disintegration and necrosis of epithelial tissue, and often pus that results from prolonged tissue ischemia caused by pressure that exceeds capillary pressure. Such ulcers are most common on weight bearing portions of the body, e.g., ischium, sacrum, trochanter, and the heel.

As used herein, “venous stasis ulcers” are understood as ulcers that arise due to poor circulation, likely due to poor valve function, causing blood to pool. Venous stasis ulcers typically occur in the legs, particularly the lower legs.

“Arterial ulcers”, also known as ischemic ulcers, are mostly located on the lateral surface of the ankle or the distal digits. The ulcers are caused by lack of blood flow to the capillary beds of the lower extremities. When pressure is placed on the skin, the skin is damaged and is unable to be repaired due to the lack of blood perfusing the tissue. The wound has a characteristic deep, punched out look, often extending down to the tendons.

As used herein, “diabetes” is understood to include all types of diabetes, e.g., type 1 and type 2 diabetes. Diabetes is a disease that results in abnormal glucose metabolism due to an insulin deficiency (both type 1 and type 2 diabetes) and/or an insensitivity to insulin (more predominant in type 2) which eventually can lead to multiple complications. Type 1 diabetes, also known as juvenile diabetes or insulin dependent diabetes tends to have an earlier onset, by young adulthood, and is predominantly an autoimmune disorder in which the pancreas is destroyed, preventing the body from making insulin. Type 2 diabetes, also known as adult onset diabetes, is a resistance to insulin that typically occurs in overweight subjects. The body produces insulin, but not in an amount sufficient to regulate blood sugar levels. Long term complications of both type 1 and type 2 diabetes include diabetic retinopathy potentially leading to blindness, development of chronic sores and infections, nerve damage and peripheral neuropathies, difficulty in controlling blood pressure and cholesterol potentially leading to cardiac problems, and kidney damage.

As used herein, a “slow healing wound” is a wound that is not fully closed three, four, five, six, seven, eight, or 9 weeks after surgery. Closure after surgery is understood as closure of the epidermis at the site of the wound when the wound is in the skin, or closure of the corresponding tissue on an internal wound. The wound may be closed by a clot or scar tissue, or a combination thereof. Closure of a wound provides a barrier to infection.

As used herein, “poorly vascularized” as in a surgical site that is poorly vascularized is understood to include tissues which innately have little vasculature, e.g., joints, ligaments, meniscus, colon, vaginal area, breast; or tissues, e.g., skin, that due to a particular disease, condition, or treatment with a drug have reduced vasculature, or vascular or circulatory deficiencies. Diseases and conditions that cause a tissue to be poorly vascularized include, but are not limited to, diabetes, rheumatoid arthritis, vascular insufficiency, vascular disease, cancer, leukemia, inflammatory diseases, or current or prior use of certain drugs including corticosteroids, drugs for the treatment of asthma/COPD, chemotherapeutic agents and anti-inflammatory agents.

Methods to Promote Bone Growth and Cartilage Repair with Systemically Administered Thrombin Derived Peptides

In one embodiment, the present invention is directed to methods of stimulating bone growth at a site in a subject in need of bone growth. The method may include administering, e.g., systemically administering, an effective amount of a thrombin peptide derivative to the subject. The site in need of bone growth may be in need of osteoinduction. A site in need of osteoinduction may be, for instance, a bone graft, a segmental gap in a bone, a bone void, at a non-union fracture, of a spinal fusion. The site in need of bone growth may be a site of a simple fracture, a site of bone surgery, a site of traumatic bone injury, or a site of distraction osteogenesis. The subject may be osteopenic, have normal bone density, or have osteoporosis. The subject may be suffering from osteogenesis imperfecta, is post-menopausal women, or be of advanced age.

In one embodiment, the present invention is directed to methods of stimulating cartilage growth or repair at a site in a subject in need of cartilage growth or repair. The method may include administering, e.g., systemically administering, an effective amount of a thrombin peptide derivative to the subject. The site in need of cartilage growth or repair may be an arthritic joint in a subject suffering from arthritis, such as rheumatoid arthritis or osteoarthritis. In one embodiment the subject is being treated for cartilage damage or loss. The cartilage damage or loss may be due to traumatic injury, or chronic injury. The subject may be a normal subject, or may be suffering from one or more conditions such as diabetes, advanced age, hypercholesterolemia, vascular disease, osteopenia, osteoporosis, and osteogenesis imperfecta. In one embodiment, the subject has not been exposed to excess radiation.

“Osteoinduction” refers to stimulating bone growth at a site within a subject at which little or no bone growth would occur if the site were left untreated. Sites which could therapeutically benefit from the induction of bone growth are referred to as “in need of osteoinduction”. Examples include non-union fractures or other severe or massive bone trauma.

It is noted that bone growth normally occurs at bone injuries such as simple or hairline fractures and well opposed complex fractures with minimal gaps without the need for further treatment. Such injuries are not considered to be “in need of osteoinduction”. Induction of bone formation required to fill non-union fractures, segmental gaps or bone voids caused, for example, by removal of a bone tumor or cyst. These cases require bone grafting or induction of new bone growth generally employing some type of matrix or scaffolding to serve as a bone growth substitute. Induced bone growth can also be therapeutically beneficial at certain sites within a subject (referred to as “ectopic” sites) where bone tissue would not normally be found, such as a site in need of a bone graft or bone fusion. Fusions are commonly used to treat lower back pain by physically coupling one or more vertebrae to its neighbor. The bone created by such a fusion is located at a site not normally occupied by bone tissue. Osteoinduction at these ectopic sites can act as a “graft substitute” whereby induced bone growth between the vertebrae takes the place of a graft and obviates the need for a second operation to harvest bone for the grafting procedure. Induction of bone growth is also needed for treating acquired and congenital craniofacial and other skeletal or dental anomalies (see e.g., Glowacki et al., Lancet 1: 959 (1981)); performing dental and periodontal reconstructions where lost bone replacement or bone augmentation is required such as in a jaw bone; and supplementing alveolar bone loss resulting from periodontal disease to delay or prevent tooth loss (see e.g., Sigurdsson et al., J. Periodontol., 66: 511 (1995)).

Simple fracture repair appears to be quite different from sites where bone formation is required to fill non-union fractures, segmental gaps or bone voids. A “simple fracture” is understood as a fracture that does not result in the formation of a gap or a misalignment of bones. For example, simple fractures include incomplete fractures, in which the bones are still partially joined, and complete fractures in which bone fragments separate completely, but in which the bones remain aligned, or are aligned by proper setting of the bones. Although such fractures typically heal in time by immobilization of the site of the fracture site, in some situations and populations, increasing the rate of healing, e.g., as demonstrated by a decrease in the amount of time required for immobilization, and/or a decrease in the amount of time to healing as determined by radiologic assessment, may be desirable even in normal individuals. Further, some subjects with particular diseases or conditions heal slowly (e.g., individuals who are not “normal”). For example, subjects with weakened or potentially weakened bones, e.g., subjects with osteopenia, osteoporosis, osteogenesis imperfecta, post-menopausal women subjects of advanced age (e.g., at least 60 years of age, at least 65 years of age, at least 70 years of age); or subjects with impaired circulation, e.g., diabetic subjects, subjects of advanced age, hypercholesterolemic subjects, subjects with vascular disease, would likely suffer from decreased ability to heal, and increased benefit from a shortened time of immobilization.

As used herein, in certain embodiments a “normal” subject is understood as a subject that does not have a disease or condition, and is not undergoing a therapeutic regimen (e.g., chemotherapy, radiation therapy, steroid treatment) that would inhibit one or more of bone growth, bone repair, cartilage growth, or cartilage repair. Such diseases and conditions that are not present in normal subjects can include osteopenia, osteoporosis, osteogenesis imperfecta, diabetes, hypercholesterolemia, and vascular disease resulting in poor circulation, or advanced age (e.g., at least 60 years of age, at least 65 years of age, at least 70 years of age). Normal subject in which an increased healing rate would be beneficial would include, for example, subjects undergoing distraction osteogenesis procedures. Increased healing rates may also be beneficial in those with complex fractures. A subject with normal bone density has a bone mineral density that is no less than 1.0 standard deviations less than the mean peak bone mass (average of young, healthy adults) as measure by Dual-energy x-ray absorptiometry (DXA).

“Osteopenia” is a condition where bone mineral density is lower than normal. It can be a precursor to osteoporosis, but does not inevitably lead to osteoporosis. Specifically, osteopenia is defined as a bone mineral density is between 1.0 and 2.5 standard deviations below the mean peak bone mass (average of young, healthy adults) as measure by DXA.

“Osteoporosis” is a disease of bones that leads to an increased risk of fracture. In osteoporosis the bone mineral density (BMD) is reduced, bone microarchitecture deteriorates, and the amount and variety of proteins in bone is altered. Osteoporosis is defined by the World Health Organization (WHO) as a bone mineral density that is 2.5 standard deviations or more below the mean peak bone mass (average of young, healthy adults) as measured by DXA. The term “established osteoporosis” includes the presence of a fragility fracture. Osteoporosis itself has no specific symptoms. Its main consequence is the increased risk of bone fractures. Osteoporotic fractures are those that occur in situations where healthy people would not normally break a bone. Typical fragility fractures occur in the vertebral column, rib, hip, and wrist.

Sites in need of cartilage growth, repair, or regeneration are found in subjects with osteoarthritis. Osteoarthritis or degenerative joint disease is a slowly progressive, irreversible, often monoarticular disease characterized by pain and loss of function. The underlying cause of the pain and debilitation is the cartilage degradation that is one of the major symptoms of the disease. Hyaline cartilage is a flexible tissue that covers the ends of bones and lies between joints such as the knee. It is also found in between the bones along the spine. Cartilage is smooth, allowing stable, flexible movement with minimal friction, but is also resistant to compression and able to distribute applied loads. As osteoarthritis progresses, surfaces of cartilage and exposed underlying bone become irregular. Instead of gliding smoothly, boney joint surfaces rub against each other, resulting in stiffness and pain. Regeneration of damaged cartilage and the growth of new cartilage at these arthritic sites would relieve the pain and restore the loss of function associated with osteoarthritis. Cartilage loss or damage can result from chronic or acute injury or insult.

For example, rheumatoid arthritis, a chronic, systemic inflammatory disorder that predominantly affects the synovial joints can also lead to the destruction of cartilage at the effected joints. Repetitive use injuries can also result in loss or damage of cartilage. Such sites are in need of and could benefit by cartilage growth and repair.

Cartilage damage can also occur from an acute insult such as trauma resulting from injury or surgery. Sports injuries are a common cause of cartilage damage, particularly to joints such as the knee. Traumatic injury to cartilage can result in the same type of functional impairment. Therefore, sites in a subject with cartilage that has been damaged by trauma or disease are in need of treatment to restore or promote the growth of cartilage.

As used herein, “poorly vascularized” as in a surgical site that is poorly vascularized is understood to include tissues which innately have little vasculature, e.g., joints, ligaments, meniscus; or tissues that due to a particular disease, condition, or treatment with a drug have reduced vasculature, or vascular or circulatory deficiencies. Diseases and conditions that cause a tissue to be poorly vascularized include, but are not limited to, diabetes, rheumatoid arthritis, vascular insufficiency, vascular disease, cancer, leukemia, inflammatory diseases, hypercholesterolemia, or current or prior use of certain drugs including corticosteroids, drugs for the treatment of asthma/COPD, chemotherapeutic agents and anti-inflammatory agents.

Methods to detect bone growth or repair are known in the art. For example, bone growth or repair can be detected by time to healing as assessed by, for example, time of immobilization required after bone damage, radiologic assessment, and range of motion assessment. Cartilage growth and repair can be assessed, for example, by pain, range of motion, and imaging methods (e.g., magnetic resonance imaging).

As used herein, a “subject that has not been exposed to excess radiation” is understood as a subject who has not been exposed to therapeutic radiation at the site in need of bone growth or repair, or accidentally exposed to excess radiation, e.g., nuclear accident or attack. As used herein, “therapeutic radiation” is understood as radiation to treat a disease or condition, e.g., cancer; or to prevent unwanted bone growth, e.g., to prevent abnormal bone growth or heterotopic ossification. As used herein, “therapeutic radiation” is distinct from “diagnostic radiation” in which the subject is exposed to a far lower level of radiation. For example, a subject with a fracture will likely be subject to radiation in the form of one or more diagnostic x-ray. However, the amount of radiation exposure from a diagnostic x-ray is less than the exposure of a subject treated with therapeutic radiation, and therefore is not considered to be “excess radiation”. Radiation therapy is typically high level focused exposure in single or “fractioned” exposures (given in lower repeated dosages to help avoid killing normal tissues). Exposures for cancer radiation therapy may be 15 to 20 Gy or more. A typical diagnostic x-ray would be less than 0.01 Gy. Approximate doses of radiation from various diagnostic, therapeutic, and environmental sources are provided in the table below:

| | Typical effective | |
|---|---|---|
| Examination | dose (mSv) | (millirem) |
| X-ray Personnel security | 0.00025 | 0.025 |
| screening scan | | |
| Chest X-ray | 0.1 | 10 |
| Head CT | 1.5 | 150 |
| Screening mammography | 3 | 300 |
| Abdomen CT | 5.3 | 530 |
| Chest CT | 5.8 | 580 |
| CT colonography (virtual | 3.6-8.8 | 360-880 |
| colonoscopy) | | |
| Chest, abdomen and | 9.9 | 990 |
| pelvis CT | | |
| Cardiac CT angiogram | 6.7-13 | 670-1300 |
| Barium enema | 15 | 1500 |
| Neonatal abdominal CT | 20 | 2000 |

(en.wikipedia.org/wiki/X-ray_computed_tomography#Typical_scan_dose)

One Gy is equal to 100 rem or 100,000 mrem. Therefore, typical chest Xray is 580 mrem or 0.0058 Gy That would be about 0.0003 of the dose used for radiation therapy. CT scan at 2000 mrem would be 0.02 Gy or about 1 one thousandth of the dosage used for radiation therapy. Therefore, a “subject that has not been exposed to excess radiation” has been exposed to less than 0.5 Gy, preferably less than 0.1 Gy of radiation, e.g., from diagnostic radiation. Natural background radiation exposure accounts for an average of 3.1 mSv/yr with variations depending on where you live. In the US, the average person is exposed to an additional 3.0 mSv/yr from medical sources (predominantly CT scans) (see the worldwide web at “xrayrisk.com.”

Further, a subject exposed to occupationally acceptable levels of radiation or naturally occurring environmental radiation is a “subject that has not been exposed to excess radiation”, i.e., a subject that has been exposed to normal radiation. In the methods provided herein, the subject is not exposed to excess radiation for at least one week prior to treatment with the peptide to promote bone or cartilage growth or repair. Preferably, the subject is not exposed to excess radiation for at least two weeks, three weeks, or four weeks prior to treatment with the peptide to promote bone or cartilage growth or repair.

A “subject that has not been exposed to excess radiation” through nuclear accident or attack is not suffering from a “radiation related injury” or a “radiation induced illness.”

As used herein, “a site in a subject in need of bone growth or repair” is understood as a site at which growth of bone is beneficial to the subject, e.g., in the healing of a fracture. Heterotypic ossification is not beneficial to the subject.

As used herein, “a site in a subject in need of cartilage growth or repair” is understood as a site at which the cartilage is damaged or insufficient such that the subject experiences pain, a reduced range of motion, or other loss of function in the joint.

As used herein, a “chronic injury” is an injury marked by long duration or frequency of recurrence, e.g., a repetitive motion injury, an injury resulting from a chronic condition, e.g., cartilage damage as a result of rheumatoid arthritis.

### Thrombin Derivative Peptides

Thrombin peptide derivatives (also: “thrombin derivative peptides”) are analogs of thrombin that have an amino acid sequence derived at least in part from that of thrombin and are active at the non-proteolytically activated thrombin receptor (NPAR). Thrombin peptide derivatives can include, for example, peptides that are produced by recombinant DNA methods, peptides produced by enzymatic digestion of thrombin, and peptides produced synthetically, which can comprise amino acid substitutions compared to thrombin and/or modified amino acids, especially at the termini.

Thrombin peptide derivatives of the present invention include thrombin derivative peptides described in U.S. Pat. Nos. 5,352,664 and 5,500,412. In one embodiment, the thrombin peptide derivatives of the present invention is a thrombin peptide derivative or a physiologically functional equivalent, i.e., a polypeptide with no more than about fifty amino acids, preferably no more than about thirty amino acids and having sufficient homology to the fragment of human thrombin corresponding to thrombin amino acids 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:6) that the polypeptide activates NPAR.

In another embodiment, the thrombin peptide derivatives of the present invention is a thrombin peptide derivative comprising a moiety represented by Structural Formula (I):

Asp-Ala-R (I).

R is a serine esterase conserved domain. Serine esterases, e.g., trypsin, thrombin, chymotrypsin and the like, have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in aspects which do not affect the function of the thrombin receptor binding domain or the serine esterase conserved amino acid sequence. Such aspects may include, but are not limited to, conservative amino acid substitutions (as defined below) and modifications, for example, amidation of the carboxyl terminus, acetylation of the amino terminus, conjugation of the polypeptide to a physiologically inert carrier molecule, or sequence alterations in accordance with the serine esterase conserved sequences.

A domain having a serine esterase conserved sequence can comprise a polypeptide sequence containing at least 4-12 of the N-terminal amino acids of the dodecapeptide previously shown to be highly conserved among serine proteases (Asp-X₁-Cys-X₂-Gly-Asp-Ser-Gly-Gly-Pro-X₃-Val; SEQ ID NO:13); wherein X₁, is either Ala or Ser; X₂ is either Glu or Gln; and X₃ is Phe, Met, Leu, His, or Val).

In one embodiment, the serine esterase conserved sequence comprises the amino acid sequence of SEQ ID NO:14 (Cys-Glu-Gly-Asp-Ser-Gly-Gly-Pro-Phe-Val) or a C-terminal truncated fragment of a polypeptide having the amino acid sequence of SEQ ID NO:14. It is understood, however, that zero, one, two or three amino acids in the serine esterase conserved sequence can differ from the corresponding amino acid in SEQ ID NO:14. Preferably, the amino acids in the serine esterase conserved sequence which differ from the corresponding amino acid in SEQ ID NO:14 are conservative substitutions as defined below, and are more preferably highly conservative substitutions. A “C-terminal truncated fragment” refers to a fragment remaining after removing an amino acid or block of amino acids from the C-terminus, said fragment having at least six and more preferably at least nine amino acids.

In another embodiment, the serine esterase conserved sequence comprises the amino acid sequence of SEQ ID NO:15 (Cys-X₁-Gly-Asp-Ser-Gly-Gly-Pro-X₂-Val; X₁ is Glu or Gln and X₂ is Phe, Met, Leu, His or Val) or a C-terminal truncated fragment thereof having at least six amino acids, preferably at least nine amino acids.

In a preferred embodiment, the thrombin peptide derivative comprises a serine esterase conserved sequence and a polypeptide having a more specific thrombin amino acid sequence Arg-Gly-Asp-Ala (SEQ ID NO:16). One example of a thrombin peptide derivative of this type comprises Arg-Gly-Asp-Ala-Cys-X₁-Gly-Asp-Ser-Gly-Gly-Pro-X₂-Val (SEQ ID NO: 1). X₁ and X₂ are as defined above. The thrombin peptide derivative can comprise the amino acid sequence of SEQ ID NO:6 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val) or an N-terminal truncated fragment thereof, provided that zero, one, two or three amino acids at positions 1-9 in the thrombin peptide derivative differ from the amino acid at the corresponding position of SEQ ID NO:6. Preferably, the amino acid residues in the thrombin peptide derivative which differ from the corresponding amino acid residues in SEQ ID NO:6 are conservative substitutions as defined below, and are more preferably highly conservative substitutions. An “N-terminal truncated fragment” refers to a fragment remaining after removing an amino acid or block of amino acids from the N-terminus, preferably a block of no more than six amino acids, more preferably a block of no more than three amino acids.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRₐR_{b}, wherein Rₐ and R_{b} are independently hydrogen, a C₁-C₁₀ substituted or unsubstituted aliphatic group, or Rₐ and R_{b}, taken together with the nitrogen to which they are bonded, form a C1-C10 non-aromatic heterocyclic group, and said N-terminal acyl group is represented by R_{c}C(0)-, wherein R_{L}. is hydrogen, a C₁-C₁₀ substituted or unsubstituted aromatic group, or a C₁-C₁₀ substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:17). Alternatively, the thrombin peptide derivative comprises the amino acid sequence of SEQ ID NO:18: Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe. The thrombin peptide derivates comprising the amino acids of SEQ ID NO:6, 17, or 18 can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acids at positions 1-9 and 14-23 in the thrombin peptide derivative can differ from the corresponding amino acid in SEQ ID NO:6. It is also understood that zero, one, two or three amino acids at positions 1-14 and 19-33 in the thrombin peptide derivative can differ from the corresponding amino acid in SEQ ID NO:18. Preferably, the amino acids in the thrombin peptide derivative which differ from the corresponding amino acid in SEQ ID NO:6 or SEQ ID NO:18 are conservative substitutions as defined below, and are more preferably highly conservative substitutions. Alternatively, an N-terminal truncated fragment of the thrombin peptide derivative having at least fourteen amino acids or a C-terminal truncated fragment of the thrombin peptide derivative having at least eighteen amino acids can be used in the methods of the present invention.

A “C-terminal truncated fragment” refers to a fragment remaining after removing an amino acid or block of amino acids from the C-terminus. An “N-terminal truncated fragment” refers to a fragment remaining after removing an amino acid or block of amino acids from the N-terminus. It is to be understood that the terms “C-terminal truncated fragment” and “N-terminal truncated fragment” encompass acylation at the N-terminus and/or amidation at the C-terminus, as described above.

A preferred thrombin peptide derivative for use in the disclosed methods comprises the amino acid sequence SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val. Another preferred thrombin peptide derivative for use in the disclosed methods comprises the amino acid sequence of SEQ ID NO:19: Asp-Asn-Met-Phe-Cys-Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe. X₁ is Glu or Gln; X₂ is Phe, Met, Leu, His or Val. The thrombin peptide derivatives of SEQ ID NO:2 and SEQ ID NO:19 can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acids, or C-terminal truncated fragments of these preferred thrombin peptide derivatives, the C-terminal truncated fragments having at least eighteen amino acids, can also be used in the disclosed method.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂ (SEQ ID NO:3). Another example of a thrombin peptide derivative comprises the amino acid sequence of SEQ ID NO:6, wherein both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acids, or C-terminal truncated fragments of TP508 (or deamide TP508), the C-terminal truncated fragments having at least eighteen amino acids.

As used herein, a “conservative substitution” in a polypeptide is the replacement of an amino acid with another amino acid that has the same net electronic charge and approximately the same size and shape. Amino acids with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acids with phenyl or substituted phenyl groups in their side chains are considered to have about the same size and shape. Listed below are five groups of amino acids. Replacing an amino acid in a polypeptide with another amino acid from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in the their side chains Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

### Modified Thrombin Peptide Derivatives

In one embodiment of the invention, the thrombin peptide derivatives are modified relative to the thrombin peptide derivatives described above, wherein cysteine residues of aforementioned thrombin peptide derivatives are replaced with amino acids having similar size and charge properties to minimize dimerization of the peptides. Examples of suitable amino acids include alanine, glycine, serine, or an S′-protected cysteine. Preferably, cysteine is replaced with alanine. The modified thrombin peptide derivatives have about the same biological activity as the unmodified thrombin peptide derivatives. See Publication No. US 2005/0158301 A1, which is hereby incorporated by reference.

It will be understood that the modified thrombin peptide derivatives disclosed herein can optionally comprise C-terminal amides and/or N-terminal acyl groups, as described above. Preferably, the N-terminus of a thrombin peptide derivative is free (i.e., unsubstituted) and the C-terminus is free (i.e., unsubstituted) or amidated, preferably as a carboxamide (i.e., —C(O)NH₂).

In a specific embodiment, the modified thrombin peptide derivative comprises a polypeptide having the amino acid sequence of SEQ ID NO:4: Arg-Gly-Asp-Ala-Xaa-X₁-Gly-Asp-Ser-Gly-Gly-Pro-X₂-Val, or a C-terminal truncated fragment thereof having at least six amino acids. More specifically, the thrombin peptide derivative comprises the amino acid sequence of SEQ ID NO:20: Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val or a fragment thereof comprising amino acids 10-18 of SEQ ID NO:20. Even more specifically, the thrombin peptide derivative comprises the amino acid sequence SEQ ID NO:5: Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val, or a fragment thereof comprising amino acids 10-18 of SEQ ID NO:5. Xaa is alanine, glycine, serine or an S-protected cysteine. X₁ is Glu or Gln and X₂ is Phe, Met, Leu, His or Val. Preferably X₁ is Glu, X₂ is Phe, and Xaa is alanine. One example of a thrombin peptide derivative of this type is a polypeptide having the amino acid sequence Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:21). A further example of a thrombin peptide derivative of this type is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22). Another example of a thrombin peptide derivative of this type is the polypeptide H-Ala-Gly-Tyr-Lys-Pro-Asp-Glu-Gly-Lys-Arg-Gly-Asp-Ala-Ser-Glu-Gly-Asp-Ser-Gly-Gly-Pro-Phe-Val-NH₂ (SEQ ID NO:30) Zero, one, two or three amino acids in the thrombin peptide derivative differ from the amino acid at the corresponding position of SEQ ID NO:4, 20, 5, 21 or 22, provided that Xaa is alanine, glycine, serine or an S-protected cysteine. Preferably, the difference is conservative as defined below.

In another specific embodiment, the thrombin peptide derivative comprises a polypeptide having the amino acid sequence SEQ ID NO:23: Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe, or a fragment thereof comprising amino acids 6-28. More preferably, the thrombin peptide derivative comprises a polypeptide having the amino acid sequence SEQ ID NO:24: Asp-Asn-Met-Phe-Xbb-Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val-Met-Lys-Ser-Pro-Phe, or a fragment thereof comprising amino acids 6-28. Xaa and Xbb are independently alanine, glycine, serine or an S-protected cysteine. X₁ is Glu or Gln and X₂ is Phe, Met, Leu, His or Val. Preferably X₁ is Glu, X₂ is Phe, and Xaa and Xbb are alanine. One example of a thrombin peptide derivative of this type is a polypeptide comprising the amino acid sequence Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe (SEQ ID NO:25). A further example of a thrombin peptide derivative of this type is the polypeptide H-Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:26). Zero, one, two or three amino acids in the thrombin peptide derivative can differ from the amino acid at the corresponding position of SEQ ID NO:23, 24, 25 or 26. Xaa and Xbb are independently alanine, glycine, serine or an S-protected cysteine. Preferably, the difference is conservative as in conservative substitutions of the thrombin peptide derivatives.

An “S-protected cysteine” is a cysteine residue in which the reactivity of the thiol moiety, —SH, is blocked with a protecting group. Suitable protecting groups are known in the art and are disclosed, for example, in T. W. Greene and P. G. M. Wuts, Protective Groups in Organic Synthesis, 3^{rd} Edition, John Wiley & Sons, (1999), pp. 454-493, the teachings of which are incorporated herein by reference in their entirety. Suitable protecting groups should be non-toxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C₁-C₅ alkyl), and S-benzyl thioethers (e.g, cysteine-S—S-t-Bu). Preferably the protective group is an alkyl thioether. More preferably, the S-protected cysteine is an S-methyl cysteine. Alternatively, the protecting group can be: 1) a cysteine or a cysteine-containing peptide (the “protecting peptide”) attached to the cysteine thiol group of the thrombin peptide derivative by a disulfide bond; or 2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

### Thrombin Peptide Derivative Dimers

In some aspects of the present invention, the thrombin peptide derivatives of the methods are thrombin peptide derivative dimers. See Publication No. US 2005/0153893, which is hereby incorporated by reference. The dimers essentially do not revert to monomers and still have about the same biological activity as the thrombin peptide derivatives monomer described above. A “thrombin peptide derivative dimer” is a molecule comprising two thrombin peptide derivatives linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention comprises the thrombin peptide derivatives described above. Specifically, thrombin peptide derivatives have less than about fifty amino acids, preferably less than about thirty-three amino acids. Thrombin peptide derivatives also have sufficient homology to the fragment of human thrombin corresponding to thrombin amino acid residues 508-530: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6) so that the polypeptide activates NPAR.

In a specific embodiment, each thrombin peptide derivative comprising a dimer comprises a polypeptide having the amino acid sequence SEQ ID NO:1: Arg-Gly-Asp-Ala-Cys-X₁-Gly-Asp-Ser-Gly-Gly-Pro-X₂-Val, or a C-terminal truncated fragment thereof comprising at least six amino acids. More specifically, each thrombin peptide derivative comprises the amino acid sequence of SEQ ID NO:6: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val, or a fragment thereof comprising amino acids 10-18 of SEQ ID NO. 5. Even more specifically, the thrombin peptide derivative comprises the amino acid sequence SEQ ID NO:2: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val, or a fragment thereof comprising amino acids 10-18 of SEQ ID NO:2. X₁ is Glu or Gln and X₂ is Phe, Met, Leu, His or Val. Preferably X₁ is Glu, and X₂ is Phe. One example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). A further example of a thrombin peptide derivative of this type is a polypeptide having the amino acid sequence H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3). Zero, one, two or three amino acids in the thrombin peptide derivative differ from the amino acid at the corresponding position of SEQ ID NO:6, 1, 2, or 3. Preferably, the difference is conservative as for conservative substitutions of the thrombin peptide derivatives.

One example of a thrombin peptide derivative dimer of the present invention is represented by Formula (IV) (core sequences disclosed as SEQ ID NO: 3):

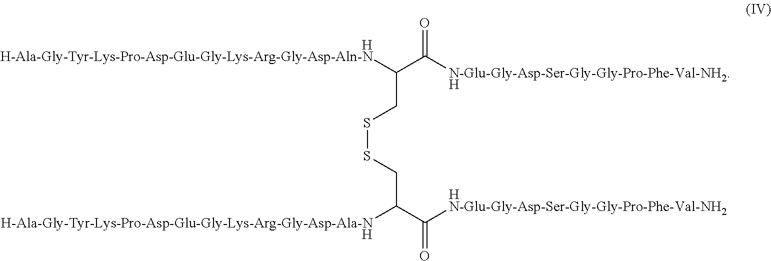

In another specific embodiment, each thrombin peptide derivative comprising a dimer comprises a polypeptide comprising the amino acid sequence SEQ ID NO:27: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr, or a C-terminal truncated fragment thereof having at least twenty-three amino acids. More preferably, each thrombin peptide derivative comprises the amino acid sequence SEQ ID NO:28: Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe-Asn-Asn-Arg-Trp-Tyr, or a C-terminal truncated fragment thereof comprising at least twenty-three amino acids. X₁ is Glu or Gln and X₂ is Phe, Met, Leu, His or Val. Preferably X₁ is Glu, and X₂ is Phe. One example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27). A further example of a thrombin peptide derivative of this type is a polypeptide comprising the amino acid sequence H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:29). Zero, one, two or three amino acids in the thrombin peptide derivative differ from the amino acid at the corresponding position of SEQ ID NO:27, 28 or 29. Preferably, the difference is conservative as defined for conservative substitutions of the thrombin peptide derivatives.

A “subject” is preferably a human, but can also be an animal in need of treatment with a thrombin peptide derivative disclosed herein, e.g., companion animals (e.g., dogs, cats, and the like), farm animals (e.g., cows, pigs, horses and the like) and laboratory animals (e.g., rats, mice, guinea pigs and the like).

An “effective amount” is the quantity of the thrombin peptide derivative described herein that results in an improved clinical outcome of the condition being treated with the thrombin peptide derivative compared with the absence of treatment. The amount of the thrombin peptide derivative administered will depend on the degree, severity, and type of the disease or condition, the amount of therapy desired, and the release characteristics of the pharmaceutical formulation. It will also depend on the subject's health, size, weight, age, sex and tolerance to drugs. Typically, the thrombin peptide derivative is administered for a sufficient period of time to achieve the desired therapeutic effect. A effective amount can be administered in one or more administrations. Typically, from about 1 μg per day to about 1 mg per day of the thrombin peptide derivatives (preferably from about 5 μg per day to about 100 mg per day) is administered to the subject in need of treatment, especially for a local means of administration. The thrombin peptide derivatives can also be administered at a dose of from about 0.1 mg/kg/day to about 15 mg/kg/day, with from about 0.2 mg/kg/day to about 3 mg/kg/day being preferred, especially for systemic means of administration. Typical dosages for the thrombin peptide derivative of the invention are also 5-500 mg/day, preferably 25-250 mg/day, especially for systemic means of administration. It is understood that the amount of peptide administered locally will depend, at least in part, on the amount of area effected (e.g., the larger the area of inflammation, the more peptide that will be administered topically). In embodiments where thrombin peptide derivatives disclosed herein are used to promote bone growth and/or cartilage repair, an effective amount can be administered at one or more times before and/or after the bone fracture or cartilage damage or growth stimulation. For example, the peptide can be administered prior to surgical bone fracture or resection (e.g., prior to surgery to remove a bone cancer, prior to distraction osteogenesis surgery, prior to spinal fusion surgery) and/or further administered throughout the time for bone healing. Similarly, cartilage growth and repair is desirable in conjunction with ligament or tendon repair surgery (e.g., anterior cruciate ligament repair, patellar tendon repair, and rotator cuff repair surgery). Prior to such surgeries, the peptides can be administered to promote cartilage growth and repair. The peptides can be used in conjunction with specific surgical techniques to stimulate cartilage growth However, it is understood that bone fractures and cartilage damage are not typically anticipated. Therefore, in some embodiments, the peptides may be administered only after the damage to the bone or cartilage has occurred.

In the methods described herein, the thrombin peptide derivative or composition can be administered before, during or after the radiation exposure. In the methods described herein, the peptide of the present invention can be administered in combination with an angiogenic growth factor. An “angiogenic growth factor” is a polypeptide which stimulates the development of blood vessels, e.g., promotes angiogenesis, endothelial cell growth, stability of blood vessels, and/or vasculogenesis. For example, angiogenic factors, include, but are not limited to, e.g., VEGF-A and members of the VEGF family, PlGF, PDGF family, fibroblast growth factor family (FGFs), TIE ligands (Angiopoietins), ephrins, ANGPTL3, ANGPTL4, etc. Angiogenic factors also include polypeptides, such as growth hormone, insulin-like growth factor-I (IGF-I), VIGF, epidermal growth factor (EGF), CTGF and members of its family, and TGF-α and TGF-β. For instance, examples of angiogenic growth factors include human VEGF-B, human VEGF-C, human VEGF-D, VEGF-E [Orfvirus (D1701)], VEGF-E [Orfvirus (NZ2)], VEGFEN27PIGF, VEGF-E/PIGF, human placental growth factor (PIGF), human platelet derived growth factor D (PDGFD), human platelet derived growth factor alpha (PDGF-a), human platelet derived growth factor 2 (PDGF2), human platelet derived growth factor C (PDGFC), angiogenin, angiopoietin-1, Del-I, acidic fibroblast growth factor (aFGF), basic fibroblast growth factor (bFGF), fibroblast growth factor 4 (FGF 4), follistatin, granulocyte colony-stimulating factor (GCSF), hepatocyte growth factor (HGF), scatter factor (SF), interleukin-8 (IL-8), leptin, midkine, placental growth factor, platelet-derived endothelial cell growth factor (PD-ECGF), plateletderived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), thymosin beta 4 (T134), connective tissue growth factor, osteopontin, and insulin growth factor (IGF-1).

The terms “treat,” “treating,” or “treatment” mean that following a period of administering the thrombin peptide derivative or composition comprising a thrombin peptide derivative, a beneficial therapeutic and/or prophylactic result is achieved, which can include a decrease in the severity of symptoms or delay in or inhibition of the onset of symptoms, increased longevity and/or more rapid or more complete resolution of the disease or condition, or other improved clinical outcome as measured according to the site that is being observed or the parameters measured for a particular disease or disorder. “Therapeutic treatment” means an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression. Treatment does not need to be curative. Treatment typically continues until the signs or symptoms of the condition are resolved as determined by the subject or a trained professional. “Prophylactic treatment” means delaying the onset, reducing the severity, and/or reducing the risk or likelihood of onset of a particular disease or condition that a subject is at risk of developing or susceptible to. Delaying can be for a short period of time (days, weeks, months) or indefinitely. Prevention can require more than one dose of the peptides provided herein. In chronic conditions, treatment can continue for an indefinite period.

In one embodiment, treatment may result in promoting bone growth or repair at a site in need of growth or repair, e.g., at a site where normal bone repair would not typically occur, or to promote cartilage growth or repair at a site in need of cartilage growth or repair, e.g., where the cartilage is damaged or deficient. Treatment also includes increasing the rate of bone growth or repair as compared to an appropriate untreated control, e.g., bone or cartilage growth or repair is no longer required.

“Reducing the risk” refers to decreasing the probability of developing a disease, disorder or medical condition, in a subject, wherein the subject is, for example, a subject who is at risk for developing the disease, disorder or condition.

“Reducing radiation related injury” refers to a decrease in the severity of injuries induced by radiation exposure.

“Diagnosing” and the like, as used herein, refers to a clinical or other assessment of the condition of a subject based on observation, testing, or circumstances for identifying a subject having a disease, disorder, or condition such as an oral complication related to treatment with a chemotherapeutic agent or radiation therapy based on the presence of at least one indicator, such as a sign or symptom of the disease, disorder, or condition such as an oral complication related to treatment with a chemotherapeutic agent or radiation therapy. Oral complications include, for example, mucositis and xerostomia. Oral complications also include one or more of oral infection, bleeding, or pain, taste alteration, nutritional compromise, and abnormal dental development. A single diagnostic test typically does not provide a definitive conclusion regarding the disease state of the subject being tested.

As used herein, “changed as compared to a control” sample or subject is understood as having at least one sign or symptom at a level that is statistically different than a sample from a normal, untreated, or control sample. Control samples include, for example, cells in culture, one or more laboratory test animals, or one or more human subjects. Methods to select and test control samples are within the ability of those in the art. Depending on the method used for detection the amount and measurement of the change can vary. Changed as compared to a control reference sample can also include a change in one or more signs or symptoms associated with or diagnostic of disease, e.g., mucositis or xerostomia. Determination of statistical significance is within the ability of those skilled in the art, e.g., the number of standard deviations from the mean that constitute a positive result.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, bucally, sublingually, transcutaneously, or mucosally. In an embodiment, an agent is administered systemically. In an embodiment, the agent is delivered locally.

As used herein, “systemic administration” is understood as various routes of administration wherein the agent is delivered to the subject in a manner that the agent is distributed throughout the organism, and is administered at a location remote from the specific site of action of the agent. As used herein, systemic administration includes, for example, non-topical, parenteral routes of administration. For example, systemic administration as used herein includes administration by injection, e.g., intravenously, subcutaneously, intramuscularly, transcutaneously, intradermally, intraperitoneally; infusion, mucosally, and intranasally. In one embodiment, the thrombin peptide derivative is administered by injection, including infusion. In one embodiment, systemic administration does not include local administration or administration of the thrombin peptide derivative directly to the site where bone growth or repair or cartilage growth or repair is to be promoted, for example, by direct contact through topical application to the site in a manner where the agent would not be distributed systemically or by use of an implant on or adjacent to the site where bone growth or repair or cartilage growth or repair is to be promoted.

As used herein, “local administration” is understood as delivery of the active agent to the site where the activity is required. Local administration may be achieved by topical administration, injection to a particular site from which the agent will not readily diffuse throughout the body (e.g., injection into a joint), or by use of a pump or implant that will provide the agent substantially to the site adjacent to the pump or implant. Topical administration includes the use of creams, lotions, ointments, liquids, or other agents that can be delivered to the site, e.g., liquids appropriate for irrigation of a surgical site. In certain embodiments, topical administration includes administration using a mouthwash or gargle, including use of a formulation containing the agent that is not intended to be swallowed. In certain embodiments, administration includes formulations with thickening agents to coat the mouth. In certain embodiments, topical administration can include the use of gums. In certain embodiments, administration includes the use of lozenges, gum, dissolvable films or tablets to be held in the mouth or under the tongue (sublingually) or bucally. Local administration to the mouth can also be achieved using oral sprays.

In one embodiment, for methods of reducing radiation related injury in a subject who is undergoing a radiation therapy, the thrombin peptide derivative is administered to normal tissue of the subject that is exposed or is to be exposed to the radiation. For example, the thrombin peptide derivative is topically administered to the normal skin that is exposed to the radiation. During a radiation therapy, radiation often causes damage to underlying tissues surrounding the target site of radiation, often because radiation exposure cannot be limited to the target site. To reduce the radiation related injury to these normal tissues, the thrombin peptide derivative can be directly applied to the underlying tissues, locally (e.g., by injection or implantation of a sustained release device and the like or through a catheter) or systemically, e.g., before, during or after the radiation therapy. Alternatively, the thrombin peptide derivative can be applied or delivered locally or systemically during the radiation therapy or after the radiation therapy. In one embodiment, local administration of the thrombin derivative peptide can be topical.

The thrombin peptide derivative can be advantageously administered in a sustained release formulation. The thrombin peptide derivative can be administered chronically, wherein the peptide derivative is administered over a long period of time (at least 60 days, but more typically, for at least one year), at intervals or by a continuous delivery method, to treat a chronic or recurring disease or condition.

The thrombin peptide derivative can be administered to the subject in conjunction with an acceptable pharmaceutical carrier as part of a pharmaceutical composition. The formulation of the pharmaceutical composition will vary according to the route of administration selected. Suitable pharmaceutical carriers may contain inert ingredients which do not interact with the compound. The earners should be biocompatible, i.e., non-toxic, non-inflammatory, non-immunogenic and devoid of other undesired reactions at the administration site. Examples of pharmaceutically acceptable carriers include, for example, saline, aerosols, commercially available inert gels, or liquids supplemented with albumin, methyl cellulose or a collagen matrix. Standard pharmaceutical formulation techniques can be employed, such as those described in Remington's Pharmaceutical Sciences, Mack Publishing Company, Easton, Pa. Other suitable pharmaceutical carriers include those described in U.S. Pat. No. 7,294,596, the entire teaching of which is incorporated herein by reference.

The compositions used in the methods of the present invention can additionally comprise a pharmaceutical carrier in which the thrombin peptide derivative is dissolved or suspended. Examples of pharmaceutically acceptable carriers include, for example, saline, aerosols, commercially available inert gels, or liquids supplemented with albumin, methyl cellulose or a collagen matrix.

Injectable delivery formulations may be administered intravenously or directly at the site in need of treatment. The injectable carrier may be a viscous solution or gel.

Delivery formulations include physiological saline, bacteriostatic saline (saline containing about 0.9% mg/mL benzyl alcohol), phosphate-buffered saline, Hank's solution, Ringer's-lactate, or liquids supplemented with albumin, methyl cellulose, or hyaluronic acid. Injectable matrices include polymers of poly(ethylene oxide) and copolymers of ethylene and propylene oxide (see Cao et al, J. Biomater. Sci 9:475 (1998) and Sims et al, Plast Reconstr. Surg. 98:843 (1996), the entire teachings of which are incorporated herein by reference).

Methods for encapsulating compositions (such as in a coating of hard gelatin or cyclodextran) are known in the art (Baker, et al, “Controlled Release of Biological Active Agents”, John Wiley and Sons, 1986).

Ointments are typically prepared using an oleaginous base, e.g., containing fixed oils or hydrocarbons, such as white petrolatum or mineral oil, or an absorbent base, e.g., consisting of an absorbent anhydrous substance or substances, for example anhydrous lanolin. Following formation of the base, the active ingredients are added in the desired concentration.

Creams generally comprise an oil phase (internal phase) containing typically fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryi sulfate; hydrophilic colloids, such as acacia colloidal clays, beegum, and the like. Upon formation of the emulsion, the active ingredients are added in the desired concentration.

Gels contain a base selected from an oleaginous base, water, or an emulsion-suspension base, as previously described. To the base is added a gelling agent which forms a matrix in the base, increasing its viscosity to a semisolid consistency. Examples of gelling agents are hydroxypropyl cellulose, acrylic acid polymers, and the like. The active ingredients are added to the formulation at the desired concentration at a point preceding addition of the gelling agent.

Delivery formulations can include formulations that are held in the mouth for a short period of time (i.e., a mouthwash) or used as a gargling solution. Such formulations are typically not formulated to be ingested in total. Such agents can be used as needed or the subject can retain the formulation in the mouth or gargle with the solution for a defined period of time a certain number of times per day as directed. The agent is delivered in a formulation for coating and to be retained in the mouth. Local administration to the mouth can also be achieved using lozenges, gum, dissolvable films or tablets including sublingual or buccal tablets, and oral sprays.

A thrombin peptide derivative can be administered to a subject alone or in combination with one or more other therapeutics, for example, a beta-blocker, an analgesic, an anti-inflammatory agent, an anti-plaque agent, an antiviral agent, or an antibiotic. Combination therapy can include co-formulation of the thrombin derived peptide with a second agent, e.g., an analgesic agent or an anti-inflammatory agent. Alternatively, combination therapy can include administration of agents separately, including administration of agents by different routes of administration.

Diseases and conditions that are treatable with the disclosed thrombin peptide derivatives are often accompanied by symptoms and infirmities such as pain and infection. For instance, dermal ulcers that are treatable with the disclosed thrombin peptide derivatives are often accompanied by symptoms and infirmities such as pain and infection. The need for bone or cartilage growth or repair that are treatable with the disclosed thrombin peptide derivatives are often accompanied by symptoms and infirmities related to poor circulation. In certain instances it may be advantageous to co-administer one or more additional pharmacologically active agents along with a thrombin peptide derivative to address such issues. For example, managing pain and inflammation may require co-administration with analgesic or an anti-inflammatory agent. Managing infection may require co-administration with antimicrobial, antibiotic, or disinfectant agents.

A thrombin peptide derivative can be administered to a subject alone or in combination with one or more other therapeutics, for example, a cholesterol-lowering agent, an anti-hypertensive agent, a beta-blocker, an anti-coagulant, a thrombolytic agent, an analgesic, an anti-inflammatory agent, an anti-plaque agent, insulin, a nitric oxide generating agent, an antiviral agent or an antibiotic. In one method, a thrombin peptide derivative can be administered to a subject in combination with arginine, for example, with arginine as an oral nutritional supplement.

In certain embodiments, the thrombin derived peptides are administered alone for the treatment of a dermal ulcer. It is understood that a subject suffering from dermal ulcers, particularly diabetic or pressure ulcers, suffers from other conditions that require therapeutic interventions. Therefore, treatment of a dermal ulcer with a thrombin peptide derivative alone does not preclude the use of other agents for treatment of the subject for other conditions, e.g., diabetes, blood pressure regulation, pain, etc. In certain embodiments, the methods can include administration of an angiogenic growth factor. An angiogenic growth factor may be administered locally, e.g., topically, or systemically.

As used herein, co-administration does not require an admixture of the agents or even that the agents are administered by the same route of administration. For example, topical, optionally local, analgesia can be used in combination with the systemically administered peptide. However, co-administration can include the preparation of an admixture of agents for delivery to the subject.

In certain embodiments, the invention includes treating a dermal ulcer in the subject with the thrombin peptide derivatives provided herein wherein the subject is not treated with an inhibitor agent such as those described in Davies et al. (PCT publication WO 0149309), which is incorporated herein by reference. As provided in WO0149309, inhibitor agents are derivable from and based on a protease inhibitor that are upregulated in a wound environment wherein the proteins have an adverse effect on wound healing such as urokinase plasminogen activator (UPA) and matrix metalloproteinase (MMP). Therefore, in a preferred embodiment, the invention herein provides methods of treatment of a dermal ulcer by administering a thrombin derived peptide to the subject, wherein inhibitors of proteases upregulated at the wound site, such as UPA inhibitors and MMP inhibitors are not administered to the subject for the treatment of the dermal ulcer.

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Sot: 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al, Synthesis, J: 315 (1992)). The teachings of these six articles are incorporated herein by reference in their entirety.

Thrombin peptide derivative dimers can be prepared by oxidation of the monomer. Thrombin peptide derivative dimers can be prepared by reacting the thrombin peptide derivative with an excess of oxidizing agent. A well-known suitable oxidizing agent is iodine.

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

An “alkyl” is a straight chain or branched saturated hydrocarbon radical. Typically, an alkyl group has from 1 to about 10 carbon atoms, preferably from 1 to about 4 carbon atoms. Exemplary alkyl groups include, but are not limited to, methyl, ethyl, n-propyl, iso-propyl, n-butyl, sec-butyl, tert-butyl, pentyl, cyclopentyl, hexyl, cyclohexyl, octyl and cyclooctyl.

The invention is illustrated by the following examples which are not intended to be limiting in any way.

### Example 1

### Effects of TP508 on Apoptosis of Human Microvascular Endothelial Cell (HMVEC) Exposed to Radiation

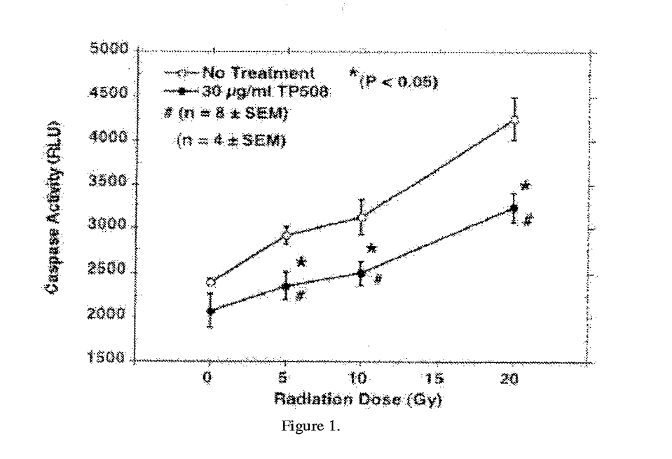

Human dermal microvascular endothelial cells (HMVEC) were irradiated using a J.L., Shepherd & Associates, Mark 1, 9K, ¹³⁷Cs Gamma Irradiator to deliver exposures of 5, 10, and 20 Gy. Following irradiation, cells received 30 u-g/ml TP508 or saline (No Treatment). After 3 h cells were assayed for activation of caspase-3 and caspase-7 as a measure of apoptosis using Caspase-Glo® 3/7 Assay (Promega, Madison, Wis.). As shown in FIG. 1, radiation caused a dose-dependent increase in Caspase 3/7 activity. TP508 treatment of these cells, however, significantly decreased radiation-induced activation of Caspase 3/7. This data demonstrates that TP508 attenuates apoptosis in microvascular endothelial cells induced by radiation.

### Example 2

### Effect of TP508 on Mouse Survival Following 8 Gy Radiation Exposure

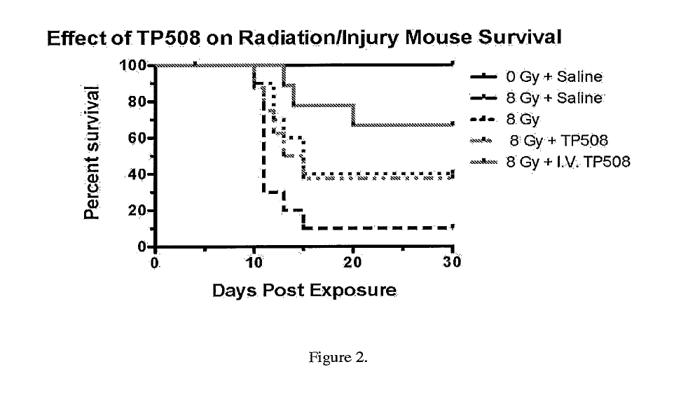

Swiss ICR mice were irradiated (¹³⁷Cs Gamma Irradiator Mark 30, Shephard and Associates, San Fernando, Calif.) with exposures of 8 Gy or 3 Gy. After 4 hours or 24 hours, mice were anesthesized and prepared for surgery. A single 1.5 cm square full dermal excision was created and treated topically with saline (25 μl) or saline plus TP508 (0.25 μg) and covered with Opsite® occlusive dressing. There was a significant decrease in survival in mice that have sustained radiation combined injuries (Table 1 below). Significant increase in survival was observed when mice were injected with TP508 24 hours after radiation exposure (see Table 1). Increase in survival was also observed in mice receiving topical treatment of wounds with TP508 (Table 1 and FIG. 2).

TABLE 1
Effect of TP508 on Survival of Mice
with Radiation Combined Injuries

| | | Mean | | Median | 30-day | |
|---|---|---|---|---|---|---|
| | | Survival | St. | Survival | survival | Signif- |
| Group | Treatment | (days) | Dev. | (days) | (%) | icance |
| 0 Gy + | Saline | 30.0 | 0 | Undefined >30 | 100 | 1 |
| Wound | | | | | | |
| 8 Gy + | Saline | 13.4 | 6.0 | 11 | 10 | 2, 3 |
| Wound | | | | | | |
| 8 Gy + | TP508 | 19.1 | 9.1 | 14 | 37 | 3 |
| Wound | Topical | | | | | |
| 8 Gy + | TP508 | 25.2 | 7.5 | Undefined >30 | 67 | 2 |
| Wound | I.V. | | | | | |
| 8 Gy | none | 21.3 | 9.3 | 15 | 40 | 1 |

1. Significant decrease in survival when radiation is combined with wounds (p = 0.0334)
2. Significant increase in survival when mice injected with TP508 24 hr after radiation exposure (p = 0.0014)
3. Increase in survival after treatment of wounds topically (NS, p = 0.1406))

### Example 3

### Effect of TP508 on Mouse Survival Following Radiation Exposure to the Lethal Dose of 12 Gy

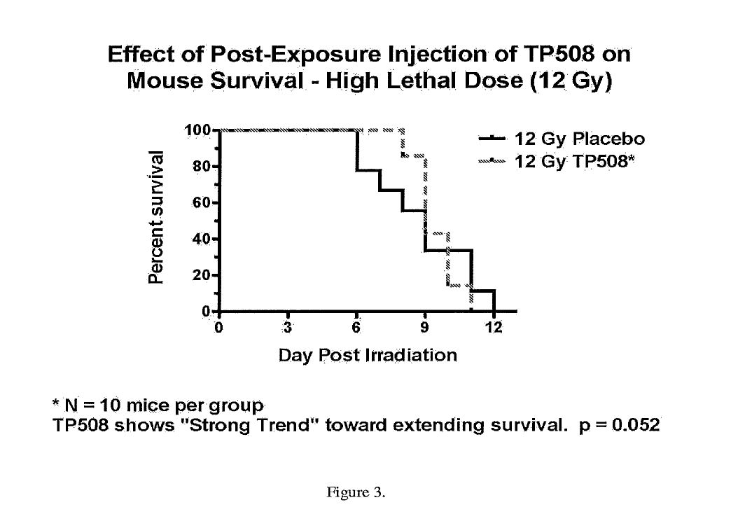

Mice were exposed to a lethal dose of ¹³⁷Cs gamma irradiation (12 Gy). Injection of a single bolus dose of TP508 (500 μg) within 2 hours after exposure delayed the mortality of the first mouse in the treated group by about 3 days and increased the group mean survival time by about 15%. (See FIG. 3). TP508 has a short half-life and may thus only be present in blood at an effective concentration for the first two to three hours. This may explain why it only extends survival for a few days.

### Example 4

### Effect of TP508 on Bacterial Growth in Blood of Animals Post Irradiation

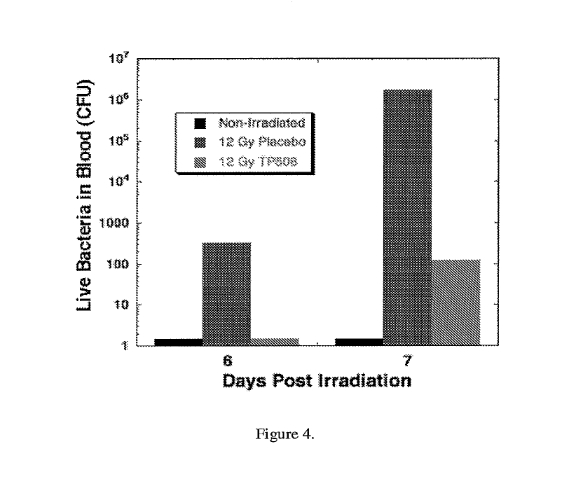

Lethal doses of radiation often cause death due to breakdown of the intestinal wall and septic infection leading to death. Therefore, the effect of TP508 to delay the onset of bacterial septic infection in irradiated mice was determined. Blood was drawn from irradiated mice (see Example 3) at various days after irradiation. Blood from each mouse (3 mice per group) was then diluted and cultured to determine the number of live bacteria quantified as colony forming units (CPU) per ml of blood. As shown in FIG. 4, by day 6 post irradiation (PI), live bacteria were present in the blood of irradiated placebo-treated mice, but not from TP508-treated mice. By day 7 the placebo-treated mice had an average of 1.6×10⁶ CFU/ml while those injected with TP508 were just beginning to show infection, with an average of just over 100 CFU/ml.

### Example 5

### Effect of TP508 on Healing of Open Dermal Wounds

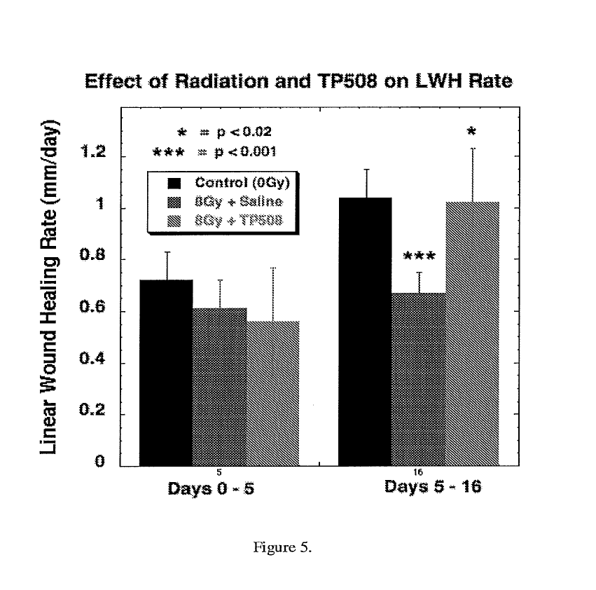

Swiss ICR mice were irradiated (¹³⁷Cs Gamma Irradiator Mark 30, Shephard and Associates, San Fernando, Calif.) with exposures of 8 Gy or 3 Gy. After 4 hours or 24 hours, mice were anesthesized and prepared for surgery. A single 1.5 cm square full dermal excision was created and treated topically with saline (25 ul) or saline plus TP508 (0.3 ug) and covered with Opsite® occlusive dressing. At 8 Gy, radiation delayed wound healing in mice receiving dermal wounds 4 hours after irradiation, but a single topical treatment with TP508 accelerated healing. The time to 50% wound closure of these wounds was 9.2 days for non-irradiated control; 13.0 days for 8 GY plus saline; and 8.9 days for 8 Gy plus TP508. Thus, TP508 appears to restore normal rates of healing to irradiated mice. This was confirmed by calculating the linear rate of healing in these wounds (See FIG. 5). Interestingly, the linear rates of healing for all groups was similar during the first 5 days after wounding, perhaps due to contraction that was not affected by radiation. From 5 to 16 days, however, radiation significantly impairs healing, but TP508 treatment overcomes this impairment.

In a second set of experiments, mice with 3 Gy exposures underwent dermal wound surgery 24 hours after irradiation. These wounds also demonstrated delayed healing relative to non-irradiated control mice. As with 8 Gy exposure experiments, TP508 topical treatment accelerated healing to overcome the effect of radiation. In these experiments, we also evaluated effects of post-exposure IV injection of TP508 on wound closure. An IV injection of TP508 about 20 hours prior to wound injury also accelerated wound closure and tended to close wounds slightly faster than topical treatment. This slight difference is also seen in comparisons of the rates of linear wound healing between non-irradiated control, 3 Gy Saline Control, 3 Gy topical TP508 and 4 Gy IV TP508. The combination of IV and topical TP508 treatment did not appear to be different than IV treatment alone.

### Example 6

### Effect of TP508 on Apoptosis and Proliferation and Migration of Intestinal Crypt Progenitor Cells

At 5 days post-exposure, histological sections of jujenum taken from mice exposed to 12 Gy whole body irradiation contain a large number of apoptotic cells within the intestinal cryps, as determined by tunnel staining. Mice injected with TP508 appear to have fewer apoptotic cells. This effect of TP508 was confirmed by measuring EdU incorporation (DNA synthesis) at 2 and 12 days post-exposure, visualizing cells that synthesized DNA during a 24-hour incubation period with Click IT®. With increasing radiation exposure fewer crypt cells continue to proliferate after 2 days. In contrast, with TP508 injection the number of cells proliferating and migrating out of the crypt with 3 Gy exposure is equivalent to non-irradiated (0 Gy) controls. In the 8 Gy sections, approximately the same number of cells are labeled, but in the TP508 group, cells tend to migrate farther up into the villi. Even after 15 Gy exposures, some crypt cells continue to proliferate in cryps of animals treated with TP508. Even 12 days after 8 Gy exposures there is decreased crypt cells proliferation and migration, yet in animals injected with TP508, the proliferation and migration appears to be fully restored to control levels.

### Example 7

### Effect of TP508 on Radiation and RCI-Induced Up-Regulation of IL-6

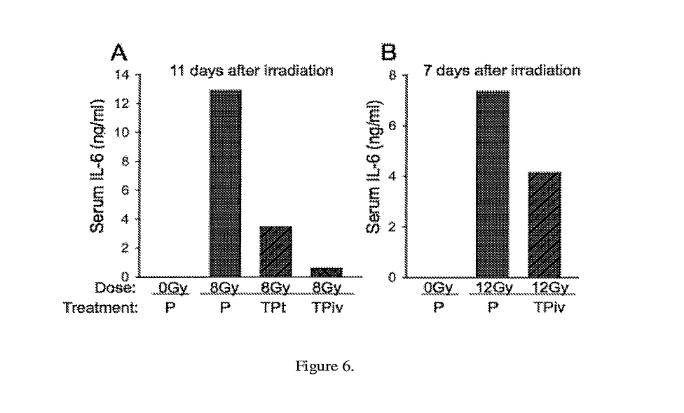

ICR white male mice were exposed to 0 Gy (control) or 8 Gy of gamma radiation and wounded 24 hours later. Wounds were treated topically with saline placebo (P) or TP508 in saline (TPt). A separate group of mice were injected IV with 500 micrograms of TP508 2 hours post 8 Gy exposure. After eleven (11) days, serum from mice was analyzed for amount of IL-6A using enzyme-linked immunosorbent assay (ELISA) (FIG. 6, Panel A).

ICR white male mice were exposed to 0 Gy, or 12 Gy nuclear irradiation without wounds and were injected IV with placebo or TP508 post-exposure. Serum was isolated from mice seven (7) days later and the amount of IL-6 was determined by enzyme-linked immunosorbent assay (ELISA) (FIG. 6, Panel B).

The combination of 8 Gy radiation exposure and wounding increases IL-6 levels above wounding alone. Topical TP508 treatment of wounds reduces IL-6 levels by ˜75%. Systemic IV injection of TP508 reduces IL-6 levels by more than 90%. 12 Gy exposure alone without wounds also increases IL-6 levels. TP508 injection reduces IL-6 production measured at day 7 by approximately 50%.

Since IL-6 increases correlate with mortality and initiation of systemic inflammatory response syndrome (SIRS) these results demonstrate that TP508 reduces systemic inflammatory response syndrome that was initiated by radiation or radiation combined with injury.

### Example 8

### Effect of TP508 on Endothelial Function as Demonstrated by Aortic Explant Endothelial Cell Sprouting Assays

To determine whether TP508 helped maintain endothelial function, an established angiogenesis assay was used. In these experiments mice were either non-irradiated or given exposures of 3 Gy, 8 Gy or 10 Gy. Approximately 2 hours post-exposure mice were injected IV with saline or saline plus TP508 (15 mg/kg). Mice were sacrificed 24 hours after exposure, aortas removed and aortic segments were placed on Matrigel® and cultured in endothelial growth medium with growth supplement containing VEGF and FGF2 for 5 days. In the non-irradiated controls (0 Gy), TP508 more than doubled the amount of endothelial sprouting from the aortic segments during 5 day incubations as determined by measuring area occupied by sprouts or longest sprout projections. Aortic segment explants from 3 Gy exposed mice had some sprouting in the saline injected group, but again this sprouting was more than doubled in mice injected with TP508. In the 8 Gy and 10 Gy groups there was virtually no sprouting from aortic segments isolated from placebo mice, while visible sprouting continued to be observed at the edges of explants from T508-treated mice.

### Example 9

### Effect of TP508 on Hematopoietic Recovery and Increases Proliferation of Bone Marrow Progenitor Cells

The bone marrow was isolated from non-exposed and mice exposed to 8 Gy with or without TP508 post-exposure injection. The samples were subjected to the complete blood count (CBC) analysis, which demonstrated earlier recovery of leukocyte, erythrocyte and thrombocyte numbers in mice treated with TP508. This result suggests that TP508 stimulates hematopoiesis or protects bone marrow cells (BMCs).

Histology of bone marrow 8 days after exposure to 8 Gy shows depletion of BMCs in marrow of 8 Gy exposed mice relative to 0 Gy mice. TP508 treatment of these mice increases number and density of BMCS.

This finding was confirmed by EdU incorporation (DNA synthesis). At 12 days post-exposure, there is some new proliferation of BMCs representing a limited degree of recovery in 8 Gy exposed mice. In TP508-treated 8 Gy mice, 3-5 times more proliferating BMCs were observed. Thus, a single post-exposure injection of TP508 initiates a cascade of events that has restorative properties for BMCs.

### Example 10

### Effect of TP508 on Bacterial Growth in Blood of Animals Post Irradiation

Lethal doses of radiation often cause death due to breakdown of the intestinal wall and septic infection leading to death. Therefore, the effect of TP508 to delay the onset of bacterial septic infection in irradiated mice was determined. Blood was drawn from mice irradiated with a lethal dose of ¹³⁷Cs gamma irradiation (12 Gy) at various days after irradiation. Blood from each mouse (3 mice per group) was then diluted and cultured to determine the number of live bacteria quantified as colony forming units (CPU) per ml of blood. By day 6 post irradiation (PI), live bacteria were present in the blood of irradiated placebo-treated mice, but not from TP508-treated mice. By day 7 the placebo-treated mice had an average of 1.6×10⁶ CFU/ml while those injected with TP508 were just beginning to show infection, with an average of just over 100 CFU/ml.

### Example 11

### Effect of TP508 on Healing of Open Dermal Wounds

Swiss ICR mice were irradiated (137Cs Gamma Irradiator Mark 30, Shephard and Associates, San Fernando, Calif.) with exposures of 8 Gy or 3 Gy. After 4 hours or 24 hours, mice were anesthesized and prepared for surgery. A single 1.5 cm square full dermal excision was created and treated topically with saline (25 ul) or saline plus TP508 (0.3 ug) and covered with Opsite® occlusive dressing. At 8 Gy, radiation delayed wound healing in mice receiving dermal wounds 4 hours after irradiation, but a single topical treatment with TP508 accelerated healing. The time to 50% wound closure of these wounds was 9.2 days for non-irradiated control; 13.0 days for 8 GY plus saline; and 8.9 days for 8 Gy plus TP508. Thus, TP508 appears to restore normal rates of healing to irradiated mice. This was confirmed by calculating the linear rate of healing in these wounds. Interestingly, the linear rates of healing for all groups was similar during the first 5 days after wounding, perhaps due to contraction that was not affected by radiation. From 5 to 16 days, however, radiation significantly impairs healing, but TP508 treatment overcomes this impairment.

In a second set of experiments, mice with 3 Gy exposures underwent dermal wound surgery 24 hours after irradiation. These wounds also demonstrated delayed healing relative to non-irradiated control mice. As with 8 Gy exposure experiments, TP508 topical treatment accelerated healing to overcome the effect of radiation. In these experiments, we also evaluated effects of post-exposure IV injection of TP508 on wound closure. An IV injection of TP508 about 20 hours prior to wound injury also accelerated wound closure and tended to close wounds slightly faster than topical treatment. This slight difference is also seen in comparisons of the rates of linear wound healing between non-irradiated control, 3 Gy Saline Control, 3 Gy topical TP508 and 4 Gy IV TP508. The combination of IV and topical TP508 treatment did not appear to be different than IV treatment alone.

### Example 12

### Effect of TP508 on Apoptosis and Proliferation and Migration of Intestinal Crypt Progenitor Cells

At 5 days post-exposure, histological sections of jujenum taken from mice exposed to 12 Gy whole body irradiation contain a large number of apoptotic cells within the intestinal cryps, as determined by tunnel staining. Mice injected with TP508 appear to have fewer apoptotic cells. This effect of TP508 was confirmed by measuring EdU incorporation (DNA synthesis) at 2 and 12 days post-exposure, visualizing cells that synthesized DNA during a 24-hour incubation period with Click IT®. With increasing radiation exposure fewer crypt cells continue to proliferate after 2 days. In contrast, with TP508 injection the number of cells proliferating and migrating out of the crypt with 3 Gy exposure is equivalent to non-irradiated (0 Gy) controls. In the 8 Gy sections, approximately the same number of cells are labeled, but in the TP508 group, cells tend to migrate farther up into the villi. Even after 15 Gy exposures, some crypt cells continue to proliferate in cryps of animals treated with TP508. Even 12 days after 8 Gy exposures there is decreased crypt cells proliferation and migration, yet in animals injected with TP508, the proliferation and migration appears to be fully restored to control levels.

### Example 13

### Clinical Trial for the Prevention of Oral Complications Using TP508 in Subjects Undergoing Treatment for Head and Neck Cancers

Subjects undergoing treatment including radiation therapy for treatment of head and neck cancers at a high risk of developing oral complications such as mucositis or xerostomia are identified using appropriate criteria, e.g., not having any prior significant dental or oral complications. Subjects are randomized into at least two groups, placebo and treatment groups. Depending on the number of subjects to be recruited, the number of treatment groups can vary.

Prior to the development of symptoms, preferably at the initiation of radiation treatment using the proper standard of care and judgment of the treating physicians, the subjects are treated with TP508 or an appropriate control. The route of delivery of TP508 can depend on the particular study. For example, the TP508 can be delivered by injection (e.g., subcutaneously, intramuscularly, or intravenously). Alternatively, subjects can be provided with a solution for gargling with and instructions for use. Subjects are monitored throughout their course of treatment for the development of signs or symptoms of oral complications, e.g., redness, inflammation, development of sores possibly with concomitant infection, difficulty speaking or swallowing, reduced or thickened saliva, etc. At the end of the course of treatment, the treatments administered to the subjects are unblinded. The presence or absence, and severity of oral complications are determined in the treatment and control groups. The results demonstrate that administration of TP508 reduces the incidence of oral complications in subjects at high risk for developing oral complications as a result of head or neck radiation as compared to an appropriate control group.

### Example 14

### Preclinical Testing of Thrombin Peptide Derivatives in Treating Mucositis in a Hamster Model Following Irradiation Therapy

The efficacy of thrombin peptide derivatives in treating oral mucositis are tested using a hamster model as disclosed in Sonis et al., Oral Oncology 36:373 (2000), the entire teachings of which are incorporated herein by reference. In this assay, mucositis is induced in the left buccal pouch of male Golden Syrian hamsters. The pouch is everted, mounted within shielding and exposed to a single dose of radiation (35-38 Gy)

TP508 is administered topically at the time of irradiation, on subsequent days, or after mucositis lesions have developed. Mucositis is scored using an established clinical scoring protocol. Pouches are everted and photographed then scored by two independent trained observers blinded from treatment groups Using the following mucositis score.

Score Description

1 Pouch completely healthy. No erythema or vasodilation. Light to severe erythema and vasodilation. No erosion of mucosa

2 Severe erythema and vasodilation. Erosion of superficial aspects of mucosa leaving denuded areas. Decreased stippling of mucosa.

3 Formation of off-white ulcers in one or more places. Ulcers may have a yellow/gray appearance due to a pseudomembrane. Cumulative size of ulcers should equal about ¼ of the pouch. Severe erythema and vasodilation.

4 Cumulative size of ulcers should equal about ½ of the pouch. Loss of pliability. Severe erythema and vasodilation

5 Virtually all of pouch is ulcerated. Loss of pliability (pouch can only partially be extracted from mouth).

A score of 1-2 is considered to represent a mild stage of the disease, whereas a score of 3-5 is considered to indicate moderate to severe mucositis.

Mucositis is alternatively, or additionally, scored using longitudinal high resolution optical Coherence tomography (OCT). A summary of this technique is described in Tearney, et al., Science 276: 2037-09 (1997) which is incorporated herein in its entirety.

### Example 15

### Methods of Treatment of Infection of Normal Wounds and Diabetic Ulcers Using TP508

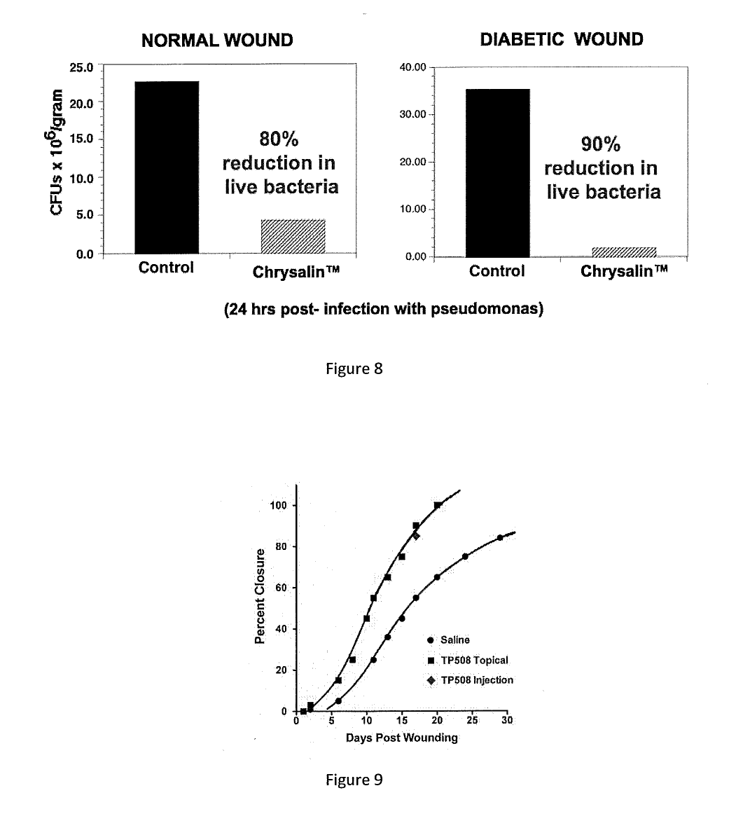

Because TP508 stimulates inflammatory cell recruitment to sites of injury, we determined whether TP508 treatment would also reduce infection in injured tissues. Full-dermal excisional wounds were created on dorsal surface of Sprague Dawley rats (2 cm diameter circles) and genetically diabetic C57BL/KsJ Lepr^{db} (db+/db+) mice (1.5 cm×1.5 cm square). Wounds were treated with saline or TP508 and 24 h later infected with Pseudomonas aeruginosa. After an additional 24 hr, the wound tissue was excised, homogenized and cultured to determine the number of live bacteria (colony forming units, CFUs). TP508 treatment reduced the number of live bacteria by 80 to 95% relative to control wounds as shown in FIG. 8.

### Example 16

### Clinical Trial for the Treatment of Oral Complications Using TP508 in Subjects Undergoing Treatment for Cancer

Subjects undergoing treatment for cancer including radiation and chemotherapy are monitored for the development oral complications. To enter the trial, the subject need not have full blown mucositis or xerostomia, but instead may have early signs or symptoms of the disease including redness, soreness, reduced or thicken saliva, difficulty eating, etc. However, subjects are recruited with various stages of oral complications that are well documented in order to determine if the complications progress, remain about the same, or regress. Subjects are further selected for inclusion in the study using appropriate criteria, e.g., not having any prior significant dental or oral complications. Subjects are randomized into at least two groups, placebo and treatment groups. Depending on the number of subjects to be recruited, the number of treatment groups can vary.

The route of delivery of TP508 can depend on the particular study. For example, the TP508 can be delivered by injection (e.g., subcutaneously, intramuscularly, or intravenously). Alternatively, subjects can be provided with a solution for gargling with and instructions for use. For subjects having identifiable areas of soreness, a topical cream or gel can be used to deliver the TP508. Subjects are monitored throughout their course of treatment for signs or symptoms of oral complications, e.g., redness, inflammation, development of sores possibly with concomitant infection, difficulty speaking or swallowing, reduced or thickened saliva, etc. At the end of the course of treatment, the treatments administered to the subjects are unblinded. A complete analysis of the subject is performed to determine of the oral complications became worse, remained about the same, or were reduced or eliminated. The results demonstrate that administration of TP508 reduces the progression of oral complications and in some cases can cause the regression of oral complications in subjects undergoing cancer treatment.

### Example 17

### Effect of TP508 on Promoting Wound Healing in Diabetic Mice

Genetically diabetic C57BL/KsJ Leprdb (db+/db+) mice were purchased from Jackson Laboratories and allowed to acclimatize prior to surgery. In a proof-of-concept experiment three mice were injected (intravenously, IV) with 100 μl of saline containing TP508 (500 μg). After 24 hours, the animals were anesthetized and 1.2 cm by 1.2 cm square full dermal excisional wounds were made on the backs of the animals. Wounds were then treated topically with saline (25 μl) and covered with occlusive dressings. The animals were housed in individual cages within an AALAC Certified Animal Care Facility at the University of Texas Medical Branch. Wounds were examined at Day 17. In two of the mice wounds were essentially closed (>95%) while one was 70% closed. Thus, average closure was 88.7% on Day 17 post surgery. Comparing this data to that from historical experiments where wounds were treated topically with saline (Saline) or 0.6 μg of TP508 (TP508) shows that the mice with TP508 injected for systemic delivery healed at a rate comparable to that of mice with TP508 topical treatment.

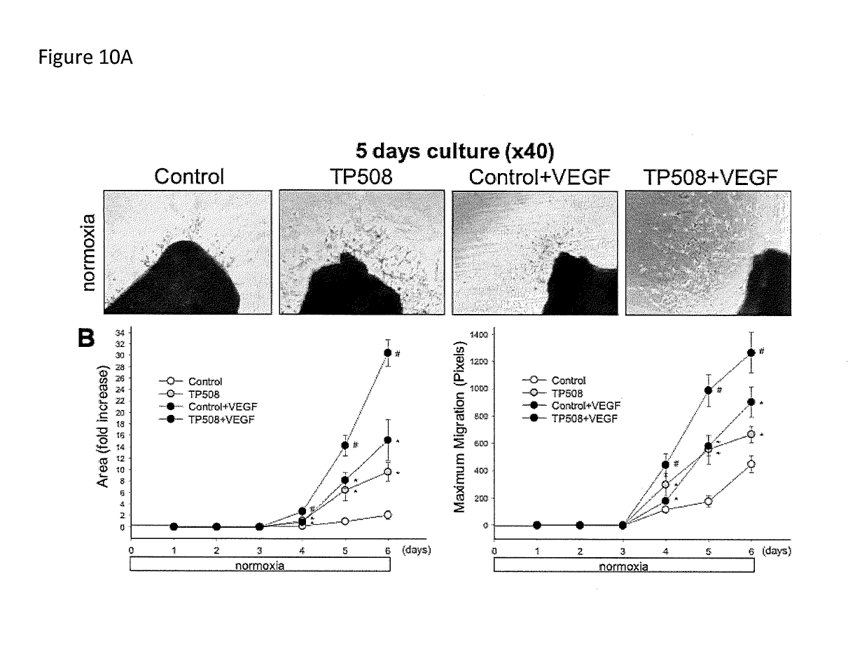

The results are shown in FIG. 9 and the table below:

| | Treatment (Historical | Days to 85% |
|---|---|---|
| Mouse Strain | Data vs. Injection) | Closure |
| Heterozygote (db/M+) | Topical saline | 9 days |
| Heterozygote (db/M+) | Topical TP508 (0.6 μg) | 8 days |
| C57BL/KjS Lepr^{db} (db+/db+) | Topical Saline | 24 days |
| C57BL/KjS Lepr^{db} (db+/db+) | Topical TP508 (0.6 μg) | 15 days |
| C57BL/KjS Lepr^{db} (db+/db+) | Intravenous Injection | 17 days |
| | TP508 (500 μg) | |

### Example 18

### Systemic Administration of Thrombin Peptide TP508 Enhances Normal and VEGF-Stimulated Angiogenesis and Attenuates Effects of Chronic Hypoxia

Revascularization and healing of chronic wounds and ischemic tissue is attenuated by the inability of angiogenic factors to stimulate angiogenesis due to ischemia or lack of endothelial cell response. The aim of this study was to investigate the systemic effect of TP508 on VEGF-stimulated angiogenesis and to evaluate the potential effect of TP508 to attenuate effects of chronic hypoxia. Systemic administration of TP508 increased endothelial sprouting from mouse aortic explants isolated 24 h after injection and potentiated VEGF-stimulated endothelial sprouting in vitro. Exposure of aortic explants to chronic hypoxia resulted in inhibition of basal and VEGF-stimulated endothelial sprouting. However, TP508 injection significantly attenuated hypoxia-induced inhibition of endothelial sprouting. These results demonstrate that TP508 systemic administration increases responsiveness of aortic endothelial cells to VEGF and diminishes the effect of chronic hypoxia on endothelial cell sprouting. These data suggest potential benefit of using a combination of systemic TP508 and local VEGF as a therapy for revascularization of ischemic tissue.

Angiogenic endothelial cell sprouting was studied by culturing mouse aortic explants on Matrigel Matrix (BD Biosciences, Bedford, Mass.). 24 h post-injection, thoracic aortas were isolated from TP508- or saline-administrated mice. The peri-aortic fibroadipose tissue was removed under a dissecting microscope and aortas were rinsed and cut transversely to create 1 mm aortic rings. Aortic rings were cut, opened, and the inner endothelial surface was placed directly on Matrigel Matrix coated wells of 24 well plates. Aortic explants were cultured in Endothelial Growth Medium (EGM) which contains EBM supplemented with 5% fetal bovine serum and SingleQuots (Lonza) in 5% CO₂ at 37° C. After 24 h culture, aortic explants from control or TP508-injected mice were stimulated with VEGF (50 ng/ml) or vehicle and cultured in normoxia. In addition, to determine effects of hypoxia, aortic explants stimulated with or without VEGF were cultured under hypoxic conditions (1% O₂, 5% CO₂) for 2 days and then switched back to normoxia.

Image analysis and quantification for area occupied by sprouting endothelial cells and maximal endothelial cell migration from aortic explants edges was performed using MetaMorph software (Molecular Devices, Downingtown, Pa.). Aortic sprouting was quantified from triplicate culture wells containing a total 6 aortic explants per experimental condition. Area of endothelial sprouting was normalized to the perimeter of aortic explants. Area of sprouting from control mice explants cultured in normoxia after 5 days was expressed as a value of 1.0. Maximum endothelial cell migration was determined by measuring the longest distance of migrated cells from aortic explants edge in 3 different regions from each explant.

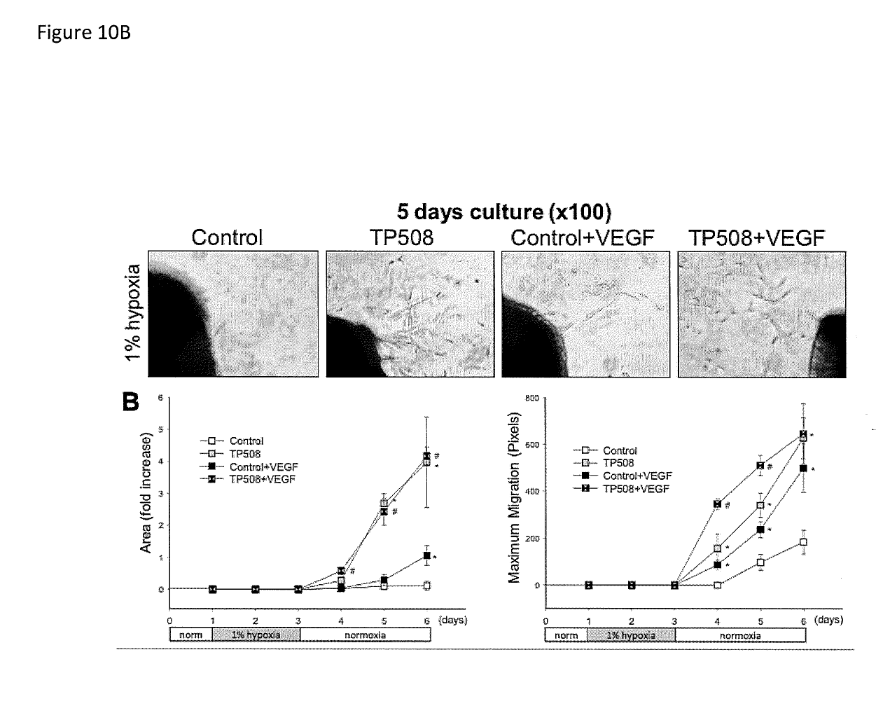

The results are shown in FIG. 10.

### Example 19

### Effect of TP508 on Promoting Pressure Ulcer Healing

Pressure ulcers can be induced using a pressure chamber with small animals and pressure cuffs in larger animals. For example, pressure wounds can be induced in pigs by mechanically applying pressure at 160-1120 mm Hg for three hours, with or without friction. Application of 70 mm Hg for longer than 2 hours. The site at which pressure is applied is also important in the induction of pressure ulcers, with muscle more susceptible to pressure damage than skin. Continuous pressure is required to cause pressure ulcers. Intermittent pressure, even at relatively high levels, does not cause the formation of pressure ulcers.

Animals are randomized into three groups. A first group is treated with TP508 prior to subjecting the animals to pressure wounding. A second group is treated with TP508 after subjecting the animals to pressure wounding. A third group is not treated with TP508 and is subjected to pressure wounding. More than three groups can be used with various dosing amounts and regimens, e.g., more than a single dose, administration of TP508 after the appearance of a wound. After wounding, the animals are observed at regularly intervals and wounds are observed and measured, preferably using quantitative methods. It is demonstrated that TP508 is useful for preventing and treating pressure wounds.

### Example 20

### Clinical Trial to Demonstrate the Efficacy of Treatment of Chronic Diabetic Ulcers with TP508

Patients with type 1 or type 2 diabetes are instructed to routinely check their feet for the presence of wounds and ulcers, and to see their physician upon discovery of such wounds as part of the standard of care for diabetes treatment. The foot wounds identified by a trained professional (e.g., physician, nurse practitioner) as being a diabetic ulcers are further classified for stage. Other pertinent medical information is also collected (e.g., age, weight, gender, duration and type of diabetes). Subjects are randomized into at least two groups, e.g., a treatment group and a control group. Depending on the number of subjects to be recruited, multiple treatment groups with various dosing amounts or regimens (e.g., number of administrations of TP508, route of administration of TP508 e.g., subcutaneously, intramuscularly, intravenously). Subjects are observed at regular intervals to determine the status of the diabetic ulcers. The study demonstrates an improved outcome for subjects treated with TP508 as compared to subjects treated with the control pharmaceutical carrier.

### Example 21

### Flow Cytometry Histograms Demonstrating the Effect of TP508 on CD45 Negative Progenitor Cell Mobilization into Blood

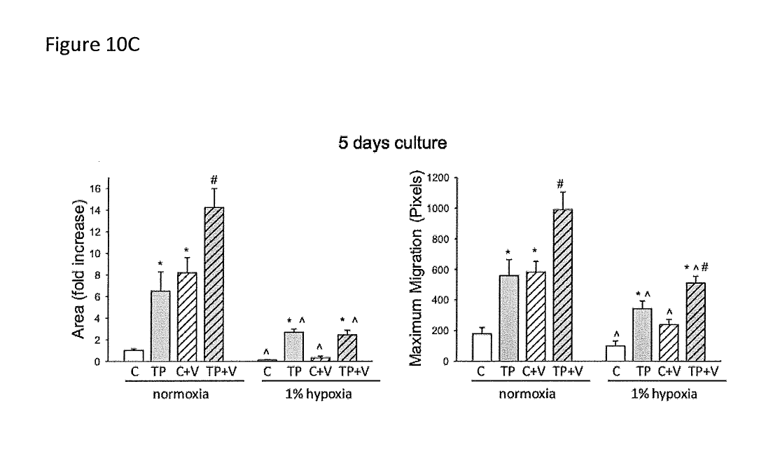

One of the markers used to identify mononuclear adult multi-potent mesynchymal stem cells (MSCs) and distinguish them from hematopoietic monocytes and lymphocytes is reduced or negative cell surface CD45 expression (Science, 1999, 284:143-147). To determine if TP508 induced mobilization of bone marrow-derived MSCs, 12 to 14 week-old ICR outbred Swiss mice were dosed with a single injection (I.P.) of TP508 (500 μg in 100 μl of sterile saline). CD45 expression in peripheral blood mononuclear cells (after removing erythrocytes) was analyzed by flow cytometry. FIG. 11 shows flow histograms comparing the number of cells vertically depending on the level of CD45 expression as determined by anti-CD45 fluorescent antibody staining. The histograms show that in control animals injected with saline alone the majority of cells expressed a high level of CD45 and the profile is represented with a single peak of cells (Panel A). In contrast, one day after TP508 injection, a second peak of cells appeared expressing much lower levels of CD45 (Panel B). Four days after injection, this second peak of cells has increased. In addition, what appears to be a third population of cells appears with little if any CD45 expression (Panel C).

These results demonstrate that injection of TP508 caused mobilization of cells expressing lower levels of CD45 from bone marrow that become a significant part of the mononuclear peripheral blood cell population within one day of injection. Release of cells with reduced CD45 continued for up to 4 days. CD45 negative MSCs have been found to be incorporated into bone (Tissue Engineering 1997, 3:173-185). Therefore, this systemic effect of TP508 on progenitor cell mobilization is expected to have a significant effect on repair of bone, cartilage, and soft tissues.

### Example 22

### Graph Depicting Changes in the Percentage of Cells Expressing Reduced Levels of CD45 Following TP508 Injection

To quantify the degree to which TP508 induced mobilization of bone marrow-derived MSCs, 12 to 14 week-old ICR outbred Swiss mice were dosed with a single injection (I.P.) of TP508 (500 μg in 100 μl of sterile saline). CD45 expression was analyzed in peripheral blood mononuclear cells (after removing erythrocytes) by flow cytometry. At indicated times (1 hr, 1, day, 2 days, 3 days, and 4 days) two mice were sacrificed and peripheral blood analyzed as demonstrated in Example 1. The area under the low level CD45 expression peak and CD45 negative peaks were determined for each mouse.

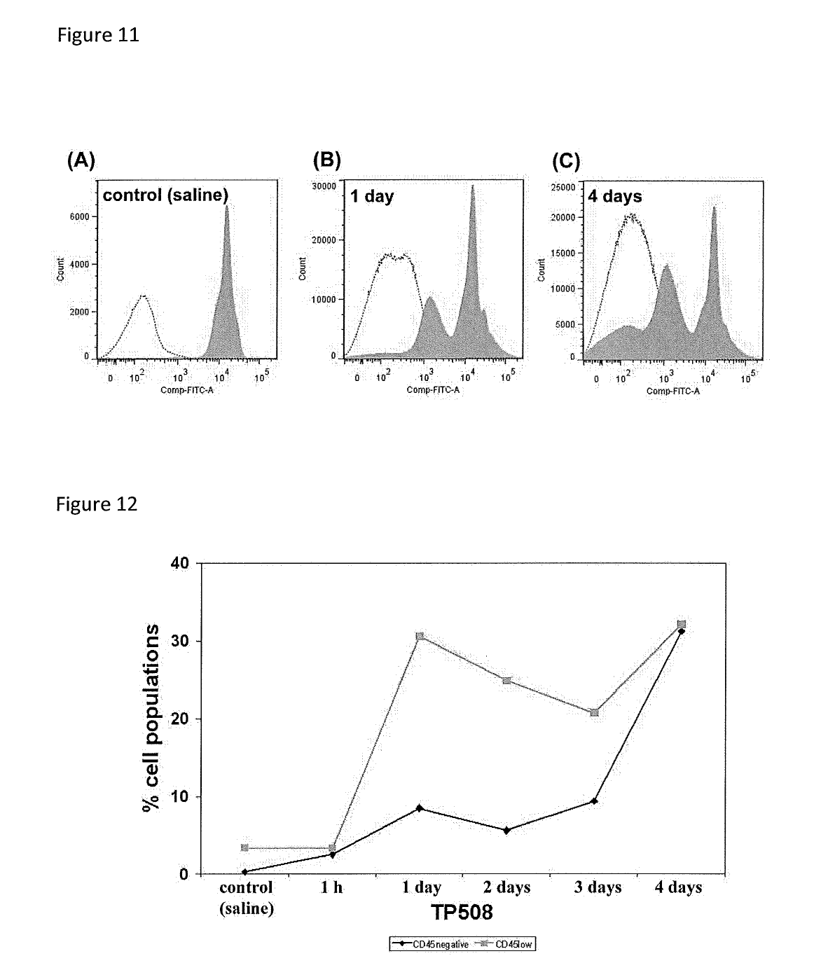

As shown in FIG. 12, by one day after TP508 injection the percentage of cells expressing low levels of CD45 increased to over 25% of total mononuclear cells and remained above 20% through day four. The percentage of cells that are CD45 negative began to increase at day one, but did not reach a level above 25% until day four.

These results suggest that a single injection of TP508 induced mobilization of bone marrow-derived mesenchymal stem cell populations that is expected to contribute to repair of soft and hard tissues.

### Example 23

### Systemically administered TP508 Induces Bone Formation in Large (1.5 cm) Defects in Rabbit Ulna

A 1.5 cm segmental defect is created in an ulna of a group of male New Zealand rabbits. A simple fracture is created in the other ulna. The rabbits are randomized into groups for treatment with various concentrations of TP508 or control (e.g., vehicle control or scrambled peptide) to demonstrate its effects on wound healing. These ulnar osteotomies are created exactly the same size by using a small metal guide to direct the cutting blade of the oscillating microsaw. TP508 was administered intravenously on a regular dosage schedule. Animals are x-rayed at two week intervals, beginning at week three, and sacrificed at nine weeks. At 9 weeks, post-surgery and the ulna-radius are removed and photographed. The healing strength of the ulna is also tested.

Ulnar osteotomies in animals treated with TP508, especially higher concentrations of TP508, show evidence of bone mineralization and growth whereas in most control osteotomies in animals not receiving TP508, there is no bone growth and/or failure to fill the voided region. Simple fractures are found to heal in both TP508 treated and untreated animals at 9 weeks, however, the simple fractures are found by x-ray to heal more quickly in animals treated with TP508. Mechanical testing for mechanical strength and stiffness (e.g., using destructive torsional testing) confirms significant effects of TP508 on bone formation in this model. Histological analysis with staining for blood vessels, collagen, bone cellularity, and other markers is also used to confirm improved healing with TP508 treatment.

### Example 24

### Systemically Administered TP508 Stimulates Bone Formation and Consolidation in Distraction Osteogenesis

Mid-tibial osteotomies are performed in a group of adult male NZW rabbits (age 24 weeks, body weight about 2.5-3.5 kg), with the tibiae stabilized with external fixators using known methods. After a 7-day latency period, once daily lengthening was initiated at rate of a 1.4 mm/day for 6 days. The rabbits are randomly divided into experimental groups. In all groups, TP508 or saline are administered systemically by intravenous injection. A range of dosages, for example, within the range of about 0.1 mg/kg/day to about 15 mg/kg/day are used, and results from the TP508-treated group were compared to a vehicle treated group.

During the experiment period, the animals are free to weight-bear on the operated leg. All animals are sacrificed at 2 weeks post-lengthening. Immediately after sacrifice, the distraction regenerate plus 5 mm of the cortical bone proximal and distal to the regenerate are excised and fixed in 95% ethanol for further examination.

Serial radiographs are taken at the day of surgery, end of lengthening, and 1 and 2 weeks post-lengthening, using a high-resolution digital radiography system. The percentage areas of the distraction gap occupied by new bone is scored by two independent and blinded observers according to the percentages of the distraction gaps filled by new bone. The percentage area of the distraction gap occupied by new bone is graded from 1 to 4 on the radiographs at 2 weeks post-lengthening. The distraction gap is considered united when bony continuity is restored across >75% of its cross-sectional area. An average score from the two observers is taken for each set of radiographs. After decoding the animal groups, the means of the scores of each group are calculated and compared.

Specimens are scanned to determine bone strength, for example using a Stratec XCT 960M (Norland Medical Systems, Fort Atkinson, Wis., USA) with the software version 5.10 (Norland Stratec Medizintechinik GmbH, Birkenfeld, Germany). Briefly prior to scanning, calibration of the pQCT is performed with a set of hydroxyapatite standards. The specimens are then placed in the holder and the centers of the regenerates are identified at the scout view window. Three slices are scanned, including the central slice, and one slice each 2 mm distal and proximal from the central slice. All slices are analyzed for total volumetric bone mineral density using the manufacturer supplied software program “XMICE v1.3”. A threshold of attenuation units is selected, based on sampling of all scans, to include mineralized tissue and exclude soft tissue. A density threshold of 275 mg/cm² is used to differentiate bone from soft tissues. The mean volumetric bone mineral densities (BMD) of the regenerates from the three slices per sample are calculated and compared.

After the pQCT examination, the samples are fixed in 10% buffered formalin for 48 h and decalcified at 4° C. over a period of 4 weeks in 14% EDTA in 0.1M Tris-HCl buffer, pH 7.2. All samples are then processed through graded alcohols, xylene and embedded longitudinally (on their coronal plane) in paraffin wax. Sections are cut at and stained with routine hematoxylin and eosin (HE) and Alcian blue/Sirius red. Alcian blue/Sirius red staining, following de-paraffin, rehydration, nuclear staining with Weigert's hematoxylin, sections are stained with Alcian blue 8GX (0.1% in 1% acetic acid) and Sirius redF3B (1% in saturated picric acid). Alcian blue stains the proteoglycanrich cartilage matrix (blue), while Sirius red stains the type I collagen fibril (red).

The study demonstrates that TP508 is useful in promoting osteogenesis in situations when augmentative treatment for bone formation and consolidation are needed

### Example 25

### Systemically Administered TP508 Stimulates Cartilage Growth in Rabbit Models

Young, male New Zealand rabbits (2-3 kilograms) (n=15) are anesthetized and given bilateral, medial longitudinal parapatellar arthrotomies. The skin, subcutaneous tissue and joint capsule are incised, using electrocautery to minimize bleeding. The joint surface is exposed by lateral dislocation of the patella. A 3-mm diameter, 1-2-mm deep full-thickness defect is made in the trochlear groove of the femur using a surgical drill and pointed stainless steel drill bit. The aim is to extend the defect into the subchondral plate without piercing the subchondral bone.

A group of the TP508-treated animals is also treated with an angiogenic growth factor, such as VEGF, after surgery at the surgical site. The growth factor is administered topically by irrigation of the surgical site.

The rabbits are divided into groups for treatment with various concentrations of TP508 or control (e.g., vehicle control or scrambled peptide) to demonstrate its effects on cartilage growth. Rabbits from each group are sacrificed at regular intervals up to 9 week when all of the animals are sacrificed. Samples are fixed and processed for histological analysis.

At the time of sacrifice, considerable fibrous granulation tissue and no evidence of white cartilage-like material are found in the control animals not treated with TP508. In contrast, in the animals treated with TP508, the defect is a nearly uniform, dense, white material filling in the defects. Different effects are observed at various time points and concentrations of TP508 with the treated animals showing greater cartilage growth at earlier time points than the control animals. Good effects are also observed in the TP508-angiogenic growth factor treated animals.

Histology of the samples are also assessed. Again, the animals treated with TP508 show a larger number of chondrocytes and signs of cartilage formation at earlier time points that the control animals. Good integration with hyaline cartilage forming at the top of the defect and extensive subchondral bone repair is also observed earlier in the TP508 treated animals. Good effects are also observed in the TP508-angiogenic growth factor treated animals.

At subsequently analyzed time points, TP508 treated defects exhibit a predominantly hyaline matrix with evidence of significant aggrecan content as shown by positive safranin-O staining, with some TP508-treated animals showing no difference in aggrecan content between the repair site and native tissue. Good effects are also observed in the TP508-angiogenic growth factor treated animals. Histological results are quantitatively assessed using a grading system such as that adapted by Freed, et al., J. Biomed. Materials Res. 28:891-899 (1944) from the scheme of O'Driscoll, et al., J. Bone Joint Surg. 126:1448-1452 (2000) with a maximum score of 25 for normal articular cartilage.

Peptide treated defects repaired with smooth articular surfaces are typically well bonded at the junction between repair and native tissue in the TP508 treated animals. The quality of control repair tissue is characterized as mostly fibrocartilage with poor quality joint surfaces. Good effects are also observed in the TP508-angiogenic growth factor treated animals. Integration at the junction between repair and native tissue is usually poor. Overall, the quality of cartilage repaired with TP508 is significantly enhanced over control non-treated defects. This improved quality of repair tissue should lead to more durable and functional restoration of joint biomechanics and reduction in the incidence of osteoarthritis in patients suffering from traumatic cartilage injuries.

All headings are for the convenience of the reader and should not be used to limit the meaning of the text that follows the heading, unless so specified.

What is claimed is:

1. A method of treating a subject exposed to a lethal dose of radiation, comprising administering to the subject an effective amount of a thrombin peptide derivative, wherein the thrombin peptide derivative comprises Asp-Ala-R, wherein R is a serine esterase conserved sequence.

2. The method of claim 1, wherein the subject is a human and the dose of radiation is at least 3.5 Gy.

3. The method of claim 1, wherein the subject has sustained a traumatic injury, a severe dermal injury, or a burn injury.

4. The method of claim 3, wherein the traumatic injury is a fractured bone or an injury to an internal organ.

5. The method of claim 3, wherein the burn injury, severe dermal injury or traumatic injury exposes the subject to systemic infection.

6. The method of claim 1, wherein the subject has hematopoietic syndrome, gastrointestinal syndrome, or brain damage.

7. The method of claim 1, wherein the thrombin peptide derivative is administered systemically.

8. The method of claim 1, wherein the thrombin peptide derivative is a polypeptide 12 to 23 amino acid residues in length.

9. The method of claim 8, wherein the serine esterase conserved sequence comprises the polypeptide of Cys-X1-Gly-Asp-Ser-Gly-Gly-Pro-X2-Val (SEQ ID NO: 15), wherein X1 is Glu or Gln and X2 is Phe, Met, Leu, His or Val.

10. The method of claim 8, wherein the thrombin peptide derivative is H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH2 (SEQ ID NO:3).

11. A method of treating a dermal ulcer in a subject, treating a wound in a subject, stimulating bone growth at a site in need of bone growth in a subject, or stimulating cartilage growth or repair at a site in a subject, comprising administering a thrombin peptide derivative to the subject, wherein the thrombin peptide derivative comprises Asp-Ala-R, wherein R is a serine esterase conserved sequence, and wherein the thrombin derived peptide is delivered systemically.

12. The method of claim 11, wherein the dermal ulcer is a diabetic ulcer, a pressure ulcer, a venous stasis ulcer, an arterial ulcer, or a chronic ulcer.

13. The method of claim 12, wherein the subject has type 1 diabetes or type 2 diabetes.

14. The method of claim 12, wherein the venous stasis ulcer is on the subject on a portion of leg below the knee.

15. The method of claim 12, wherein the arterial ulcer is on the subject on a lateral surface of the ankle or the distal digits.

16. The method of claim 11, wherein the wound is a surgical wound.

17. The method of claim 11, wherein the wound is a slow healing wound.

18. The method of claim 17, wherein the slow healing wound is a wound that is not fully closed three weeks after surgery.

19. The method of claim 11, wherein the site in need of bone growth is in need of osteoinduction, a site of a simple fracture, a site of bone surgery, a site of traumatic bone injury, or a site of distraction osteogenesis.

20. The method of claim 19, wherein the site in need of osteoinduction is a bone graft, a spinal fusion, or a segmental gap in a bone, a bone void, or at a non-union fracture.

21. The method of claim 11, wherein the subject is osteopenic, has osteoporosis, or a combination thereof.

22. The method of claim 11, wherein the method further comprises administering therapeutic radiation.

23. The method of claim 11, wherein the thrombin peptide derivative is a polypeptide 12 to 23 amino acid residues in length.

24. The method of claim 23, wherein the serine esterase conserved sequence comprises the polypeptide of Cys-X1-Gly-Asp-Ser-Gly-Gly-Pro-X2-Val (SEQ ID NO: 15), wherein X1 is Glu or Gln and X2 is Phe, Met, Leu, His or Val.

25. The method of claim 23, wherein the thrombin peptide derivative is H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH2 (SEQ ID NO:3).

26. A method of prevention or treatment of mucositis in a subject treated with a chemotherapeutic agent or radiation comprising administering a thrombin peptide derivative to the subject, wherein the thrombin peptide derivative comprises Asp-Ala-R, wherein R is a serine esterase conserved sequence.

27. The method of claim 26, wherein the thrombin peptide derivative is administered topically.

28. The method of claim 26, wherein the thrombin peptide derivative is a administered in a formulation selected from a mouthwash, a gargle, a lozenge, a gum, a dissolvable film, a dissolvable tablet, and an oral coating formulation.

29. The method of claim 26, wherein the thrombin peptide derivative is a polypeptide 12 to 23 amino acid residues in length.

30. The method of claim 29, wherein the serine esterase conserved sequence comprises the polypeptide of Cys-X1-Gly-Asp-Ser-Gly-Gly-Pro-X2-Val (SEQ ID NO: 15), wherein X1 is Glu or Gln and X2 is Phe, Met, Leu, His or Val.

31. The method of claim 29, wherein the thrombin peptide derivative is H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH2 (SEQ ID NO:3).
